(19) United States
(12) Patent Application Publication
(10) Pub. No.: US 2024/0415956 A1
(43) Pub. Date: Dec. 19, 2024

(54) COMPOSITIONS AND METHODS FOR ENHANCING EFFICACY AND REDUCING ADVERSE EFFECTS FROM COVID VACCINATION

(71) Applicants: The Chinese University of Hong Kong, Shatin, New Territories, Hong Kong SAR (CN); Versitech Limited, Hong Kong SAR (CN)

(72) Inventors: Siew Chien NG, Hong Kong SAR (CN); Ka Leung Francis CHAN, Tai Po, N.T., Hong Kong SAR (CN); Hein Min TUN, Tseung Kwan O, Hong Kong SAR (CN)

(21) Appl. No.: 18/704,050

(22) PCT Filed: Oct. 27, 2022
(86) PCT No.: PCT/CN2022/127907

§ 371 Date: Apr. 23, 2024

Related U.S. Application Data
(60) Provisional application No. 63/273,088, filed on Oct. 28, 2021.

(51) Int. Cl. A61K 39/39; A61K 35/00; A61K 35/741; A61K 35/745; A61K 39/00; A61K 45/06; A61P 37/04

(52) U.S. Cl. CPC A61K 39/39; A61K 35/741; A61K 35/745; A61K 45/06; A61P 37/04; A61K 2035/115; A61K 2039/52

(57) ABSTRACT

Probiotic compositions and methods for enhancing efficacy of COVID-19 vaccination or for reducing adverse effects of COVID-19 vaccination.

### RELATED APPLICATIONS

This application claims priority to U.S. Provisional Patent Application No. 63/273,088, filed Oct. 28, 2021, the contents of which are hereby incorporated by reference in the entirety for all purposes.

### BACKGROUND OF THE INVENTION

In recent years, viral and bacterial infection is becoming more prevalent worldwide and presents a serious public health threat. For example, the Coronavirus-2019 (COVID-19) global pandemic of a respiratory disease caused by severe acute respiratory syndrome coronavirus 2 (SARS-COV-2) has affected more than 621 million people worldwide, including over 6.5 million deaths, and is exacerbated by a lack of officially approved therapeutics as well as a lack of thoroughly tested, proven safe and effective vaccines. Several promising therapeutic agents are currently undergoing active investigation and development for prophylactic or therapeutic use in the treatment for COVID-19 to prevent or ameliorate its damaging effects to the afflicted patients, while in the meantime experimental vaccines are widely distributed to the general population.

Accordingly, there exists an urgent need for new and meaningful methods to facilitate vaccination efforts by way of enhancing vaccine safety and efficacy and/or reducing any potential adverse effects from vaccination to achieve reduction or elimination of viral and bacterial infections as well as to lessen or eliminate the downstream effects associated such infections. The purpose of this study is to identify gut microbial species that can enhance the beneficial effects of vaccination among vaccine recipients as well as to identify gut microbial species that can reduce or eliminate the detrimental or adverse effects of vaccination among vaccine recipients. Direct supplementation of these beneficial gut microbial species in various combinations to an individual who has been vaccinated recently or is expected to receive vaccination shortly is a potentially effective means to improve a person's immunity against an infectious pathogen upon vaccination against infections including COVID-19, thus preventing the development of such infectious diseases and/or reducing the severity of such diseases. On the other hand, direct supplementation of these beneficial gut microbial species in various combinations to a vaccine recipient is also a useful means for reducing the risk of undesirable outcome/adverse events stemmed from vaccination. The present invention fulfills this and other related needs by identifying beneficial gut microorganisms as well as illustrating their use in vaccination efforts and therefore in the prevention of diseases and conditions caused by viral or bacterial infections.

### BRIEF SUMMARY OF THE INVENTION

The present inventors discovered in their studies the certain gut microbial species can facilitate enhancing the desirable positive effects (e.g., immunity against SARS-COV2 infection or protection against severe illness) from COVID vaccination effects while reducing the undesirable negative effects (e.g., adverse events, both in number and severity) of various types of vaccines. The microorganisms so identified now serve to provide new methods and compositions as an integral part of the COVID-19 vaccination efforts.

In a first aspect, the present invention provides several methods for enhancing immunity (e.g., antibody response) and/or for reducing potential adverse effects from a COVID-19 vaccine in a human vaccinee. In one method for enhancing the immunity induced by vaccination, a composition, which comprises or consists essentially of an effective amount of (i) bacterial species Bifidobacterium adolescentis, or (ii) one or more of the bacterial species set forth in Table 1, plus one or more physiologically acceptable excipients, is introduced into the gastrointestinal tract of a subject receiving an inactivated vaccine such as the vaccine known as Sino Vac-Corona Vac. In some embodiments, the composition comprises no detectable amount of one other Bifidobacterium species or no detectable amount of two other Bifidobacterium species.

In another method for enhancing the immunity induced by vaccination, an obese or overweight human subject receiving an inactivated COVID-19 vaccine (such as SinoVac-CoronaVac) is given a composition comprising or consisting essentially of an effective amount of one or more of the bacterial species set forth in Table 2 along with one or more physiologically acceptable excipients, the composition being introduced into the subject's gastrointestinal tract. In some embodiments, the composition comprises or consists essentially of an effective amount of one or more of the bacterial species set forth in Table 1 or 2 plus the excipient(s).

In yet another method for enhancing the immunity induced by vaccination, a human subject having been or soon to be vaccinated with an mRNA COVID-19 vaccine (such as the BioNTech mRNA BNT162b2 vaccine) receives a composition comprising or consisting essentially of an effective amount an effective amount of (i) bacterial species Bifidobacterium adolescentis; or (ii) bacterial species Roseburia faecis; or (iii) one or more of the bacterial species set forth in Table 3 or 4; or (iv) menaquinols, plus one or more physiologically acceptable excipients, which composition is introduced into the subject's gastrointestinal tract.

In a further method for enhancing the immunity induced by vaccination, an obese or overweight human subject receiving an mRNA COVID-19 vaccine (e.g., the BioNTech BNT162b2 vaccine) has a composition comprising or consisting essentially of an effective amount of one or more of the bacterial species set forth in Table 5 plus one or more physiologically acceptable excipients introduced into his gastrointestinal tract. In some cases, the composition comprises or consists essentially of an effective amount of one or more of the bacterial species set forth in Tables 3 and 5, or Tables 4 and 5, or Tables 3, 4, and 5, plus the excipient(s).

In one method of reducing adverse effects from COVID vaccination in a human subject receiving an inactivated COVID-19 vaccine, such as the Sino Vac-Corona Vac, a composition is introduced into the subject's gastrointestinal tract: which composition comprising, or consisting essentially of, an effective amount of one or more of the bacterial species selected from Prevotella copri (NCBI: txid 165179), Megamonas funiformis (NCBI: txid 437897), Megamonas hypermegale (NCBI: txid 158847), and those in Table 6, in addition to one or more physiologically acceptable excipients.

In one method of reducing adverse effects from COVID vaccination in a human subject receiving an mRNA COVID-19 vaccine, such as the BioNTech mRNA BNT162b2vaccine, a composition is introduced into the subject's gastrointestinal tract: which composition comprising, or consisting essentially of, an effective amount of one or more of the bacterial species selected from Prevotella copri (NCBI: txid 165179), Megamonas funiformis (NCBI: txid 437897), and Megamonas hypermegale (NCBI: txid 158847), in addition to one or more physiologically acceptable excipients.

Certain additional features may be included in any one of the above-mentioned methods: in some cases the introducing step comprises delivery of the composition to the small intestine, ileum, or large intestine of the subject. In some cases, a prebiotic or therapeutic agent for COVID-19 is introduced concurrently with the composition, for example, it may be contained in the same composition or it may be administered in a separate composition. In some cases, the introducing step comprises oral ingestion of the composition(s), which may be formulated in the form of a powder, liquid, paste, cream, tablet, or capsule, for example. In some cases, the introducing step comprises direct deposit of the composition to the subject's gastrointestinal tract (e.g., small intestine, ileum, or large intestine), thus the composition(s) formulated accordingly. In some cases, the subject has received the vaccine within the past 24-48 hours from the time of receiving the composition of the present invention, or the subject is to receive the vaccine within the next 24-48 hours. In some cases, the composition of the present invention consists essentially of the specified bacterial species and one or more physiologically acceptable excipients. In some cases, the composition comprises no detectable amount of any other unnamed Bifidobacterium species, or the composition may contain only one but not two other unnamed Bifidobacterium species in any detectable amount.

In the second aspect, the present invention provides a composition for use in enhancing immunity or reducing adverse effects from COVID-19 vaccination in a subject comprising, or consisting essentially of, an effective amount of (1) any one or more bacterial species selected from Tables 1, 2, 5, and 6, Bifidobacterium adolescentis, Roseburia faecis, Prevotella copri, Megamonas funiformis, and Megamonas hypermegale; and (2) one or more physiologically acceptable excipients. In some embodiments, the composition comprises, or consists essentially of, an effective amount of (1) any one or more bacterial species selected from Table 1 and (2) one or more physiologically acceptable excipients. In some embodiments, the composition comprises, or consists essentially of, an effective amount of (1) any one or more bacterial species selected from Table 2, or any one or more bacterial species selected from Tables 1 and 2 combined, and (2) one or more physiologically acceptable excipients. In some embodiments, the composition comprises, or consists essentially of, an effective amount of (1) any one or more bacterial species selected from Tables 3 and 4 combined, and (2) one or more physiologically acceptable excipients. In some embodiments, the composition comprises, or consists essentially of, an effective amount of (1) any one or more bacterial species selected from Table 5, or any one or more bacterial species selected from Tables 3, 4, and 5 combined, and (2) one or more physiologically acceptable excipients. In some embodiments, the composition comprises, or consists essentially of, an effective amount of (1) any one or more bacterial species selected from Table 6, and (2) one or more physiologically acceptable excipients. In some embodiments, the composition comprises, or consists essentially of, an effective amount of Prevotella copri, Megamonas funiformis, and/or Megamonas hypermegale plus one or more physiologically acceptable excipients. In some embodiments, the composition is formulated for oral ingestion, e.g., in the form of a food or beverage item. In some embodiments, the composition is formulated for direct deposit to the subject's gastrointestinal tract (e.g., small intestine, ileum, or large intestine). In some embodiments, the composition may optionally further include one or more prebiotic or therapeutic agent for COVID-19.

In a third aspect, the present invention provides a kit useful for promoting efficacy of COVID-19 vaccination by enhancing efficacy/immunity or reducing adverse effects from a COVID vaccine, including a vaccine in the nature of an inactivated SARS-COV2coronavirus or a DNA-or RNA-based vaccine. The kit includes a plurality of containers, each containing a composition comprising an effective amount of one or more bacterial species selected from Tables 1, 2, 5, and 6, Bifidobacterium adolescentis, Roseburia faecis, Prevotella copri, Megamonas funiformis, and Megamonas hypermegale. In some cases, each of the compositions consists essentially of the bacterial species plus one or more physiologically acceptable excipients. In some embodiments, the compositions comprise no detectable amount of another Bifidobacterium species. In some embodiments, the kit further comprises one or more compositions each comprising an effective amount of one or more different bacterial species selected from Table 3 or 4. In some embodiments, the kit further comprises one or more compositions each comprising an effective amount of a prebiotic or therapeutic agent for COVID-19. In some embodiments, the compositions are formulated for oral ingestions, e.g., in the form of a powder, liquid, paste, cream, tablet, or capsule. In some embodiments, the compositions are formulated for direct deposit into the gastrointestinal tract (e.g., small intestine, ileum, or large intestine) of a recipient. In some embodiments, the composition does not comprise a detectable amount of another one or another two unnamed Bifidobacterium species.

### BRIEF DESCRIPTION OF THE DRAWINGS

FIG. 1 Study design and changes in beta diversity. alpha diversity and bacterial species after completion of vaccination. FIG. 1a, Study design. FIG. 1b, Beta diversity were significantly different between baseline and one month after completion of vaccination, and the changes were not different between the two vaccine groups. P values were given by PERMANOVA and Wilcoxon rank-sum test, and adjusted by FDR, respectively. FIG. 1c, Alpha diversity decreased significantly from baseline to one month after completion of vaccination. P values were given by paired Wilcoxon rank-sum test. FIG. 1d, Differentially abundantly species between baseline and one month after completion of vaccination. Differentially abundantly species were detected using Paired Wilcoxon rank-sum test (FDR corrected P value<0.05).

FIG. 2 Baseline gut microbial and functional biomarkers for high-responders vs. low-responders to vaccines. FIG. 2a, Microbial and functional biomarkers for high-responders among Corona Vac vaccinees (sVNT-10>60%). Only pairwise correlations with an FDR corrected P value<0.05 were shown. FIG. 2b, Microbial and functional biomarkers for highest-tier responders among BNT162b2 vaccinees (the first quartile of sVNT %). FIG. 2c, AUC of individual and combined biomarkers for high-responders among Corona Vac vaccinees. FIG. 2d, AUC of individual and combined biomarkers for highest-tier responders among BNT162b2 vaccines.

FIG. 3 Species contributing to the gut bacterial motility and its association with neutralizing immunity to BNT162b2 vaccine. FIG. 3a, Heatmap showing correlation between immune responses and the overall as well as detailed bacterial motility. FIG. 3b, Positive association between gut bacterial motility and sVNT readouts in BNT162b2 vaccinees. FIG. 3c, Positive association between fimbriae expressed by gut bacteria and sVNT readouts in BNT162b2 vaccinees. Correlation between motility and sVNT data was examined using Spearman's correlation test. Comparison between high-vs. low-responder groups/highest-tier vs. others was made using Wilcoxon's rank-sum test.

FIG. 4 Weight status modifies effects of beneficial bacteria on immune response in Corona Vac vaccinees. Immune response and odds ratios of becoming high-responders separated by bacterial abundance within weight strata, FIG. 4a, by Bifidobacterium adolescentis abundance. FIG. 4b, by Butyricimonas virosa abundance. FIG. 4c, by Adlercreutzia equolifaciens abundance. FIG. 4d, by Asaccharobacter celatus abundance. Comparison between NW and OWOB was done using Wilcoxon test; comparisons between subgroups were done using Dunn's test with FDR correction. Reference group: NW with high bacterial abundance. Model 1: crude model. Model 2: adjusted for age.

FIG. 5 Immune response against SARS-COV-2 in the study cohort. FIG. 5a, % inhibition to SARS-COV-2 (10-fold dilution) at baseline and at one month after the second dose of vaccine. FIG. 5b, % inhibition to SARS-COV-2 (200-fold dilution) at one month after the second dose of vaccine among BNT162b2 vaccinees. FIG. 5c, RBD-specific IgG titre (AUC) at baseline and at one month after the second dose of vaccine. FIG. 5d, Correlation between % inhibition (sVNT, 10-fold dilution) and RBD-specific IgG titre at one month in Corona Vac vaccinees. FIG. 5e, Correlation between % inhibition (sVNT, 200-fold dilution) and RBD-specific IgG titre at one month in BNT162b2 vaccinees.

FIG. 6 Gut microbiota at one month after the second dose of vaccine enriched in high-responders. FIG. 6a, Biomarkers for Corona Vac vaccinees. FIG. 6b, Biomarkers for BioNTech vaccinees.

FIG. 7 Gut microbiota dysbiosis in the subject with very low level of sVNT against BNT162b2 vaccine at FIG. 7a, phylum and FIG. 7b, species levels. Inner and outer circles in a represent BNT162b2 low-responder and others, respectively.

FIG. 8 Heatmap showing relative abundance of contributing species to gut bacterial motility in BNT162b2 vaccinees.

FIG. 9 Heatmap showing relative abundance of species significantly correlated with bacterial-type flagellum-dependent cell motility (GO:0071973) in BNT162b2 vaccinees. P value of Spearman correlations: ***, P<0.001; **, P<0.01; *, P<0.05.

FIG. 10 Heatmap showing relative abundance of species significantly correlated with bacterial fimbriae (GO:0009289) in BNT162b2 vaccinees. P value of Spearman correlations: ***, P<0.001; **, P<0.01; *, P<0.05.

FIG. 11 Baseline microbial biomarkers enriched in high-responders of Corona Vac vaccine with BMI≥23.

FIG. 12 Normalized proportion change of observed species between the baseline and one month after the second dose of BNT162b2 is associated with adverse events after the first dose.

FIG. 13 Clustering of baseline gut microbiome samples. FIG. 13a, Calinski-Harabasz index of clustering in CoronaVac vaccinees. FIG. 13b, Average sihouette width of clustering in Corona Vac vaccinees. FIG. 13c, Two clusters of Corona Vac vaccinees. FIG. 13d, Biomarkers of clusters of Corona Vac vaccinees. FIG. 13e, Calinski-Harabasz index of clustering in BNT162b2 vaccinees. FIG. 13f, Average sihouette width of clustering in BNT162b2 vaccinees. FIG. 13g, Two clusters of Corona Vac vaccinees. FIG. 13h, Biomarkers of clusters of BNT162b2 vaccinees. Clustering was based on JSD dissimilarity. Biomarkers were identified using LEfSe.

### DEFINITIONS

As used herein, the term “SARS-COV-2 or severe acute respiratory syndrome coronavirus 2,” refers to the virus that causes Coronavirus Disease 2019 (COVID-19). It is also referred to as “COVID-19 virus.”

The terms “inactivated COVID-19 vaccine” and “RNA-based COVID-19vaccine” are used to refer to COVID-19 vaccines produced by inactivating one or more strains of SARS-COV2 coronaviruses and by recombinantly generating an RNA molecule encoding a viral antigen derived from SARS-COV2 coronavirus, respectively. Examples of inactivated COVID-19 vaccine include Sino Vac-Corona Vac and Sinopharm, and examples of RNA-based COVID-19 vaccine include the RNA vaccine BNT162b2 produced by BioNTech (COMIRNATY) and the RNA vaccine produced by Moderna (mRNA-1273).

The term “inhibiting” or “inhibition,” as used herein, refers to any detectable negative effect on a target biological process, such as RNA/protein expression of a target gene, the biological activity of a target protein, cellular signal transduction, cell proliferation, presence/level of an organism especially a micro-organism, any measurable biomarker, bio-parameter, or symptom (including any adverse events) in a subject, and the like. Typically, an inhibition is reflected in a decrease of at least 10%, 20%, 30%, 40%, 50%, 60%, 70%, 80%, 90% or greater in the target process (e.g., a subject's bodyweight, or the blood glucose/cholesterol level, or any measurable symptom or biomarker in a subject, such as an infection rate among subjects by a pathogenic infectious agent, or the number or frequency of certain definable adverse events), or any one of the downstream parameters mentioned above, when compared to a control. “Inhibition” further includes a 100% reduction, i.e., a complete elimination, prevention, or abolition of a target biological process or signal. The other relative terms such as “suppressing.” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or parameter.

The term “obese” or “obesity,” as used herein, describes anyone with a body mass index (BMI) greater than or equal to 25 kg/m², whereas the term “overweight” describes anyone with a BMI greater than 23 kg/m² and less than 25 kg/m².

The term “menaquinol” refers to a quinol derived from a menaquinone, which has the following chemical structure:

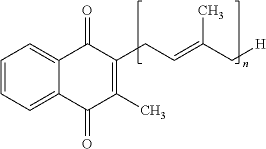

As used herein, the term “treatment” or “treating” includes both therapeutic and preventative measures taken to address the presence of a disease or condition or the risk of developing such disease or condition at a later time. It encompasses therapeutic or preventive measures for alleviating ongoing symptoms, inhibiting or slowing disease progression, delaying of onset of symptoms, or eliminating or reducing side-effects caused by such disease or condition. A preventive measure in this context and its variations do not require 100% elimination of the occurrence of an event; rather, they refer to a suppression or reduction in the likelihood or severity of such occurrence or a delay in such occurrence.

The term “severity” of a disease refers to the level and extent to which a disease progresses to cause detrimental effects on the well-being and health of a patient suffering from the disease, such as short-term and long-term physical, mental, and psychological disability, up to and including death of the patient. Severity of a disease can be reflected in the nature and quantity of the necessary therapeutic and maintenance measures, the time duration required for patient recovery, the extent of possible recovery, the percentage of patient full recovery, the percentage of patients in need of long-term care, and mortality rate.

A “patient” or “subject” receiving the composition or treatment method of this invention is a human, including both adult and juvenile human, of any age, gender, and ethnic background, who is not currently diagnosed with COVID-19 (e.g., does not have a positive nucleic acid test result for SARS-COV2) but might be at risk of being exposed to SARS-CoV2 and subsequently becoming infected, although who may have been previously diagnosed with COVID-19 (e.g., had previously had a positive nucleic acid or antibody test report for SARS-COV2 at least 4 weeks prior but has since had at least one negative nucleic acid test report), and who is soon to receive or has just received COVID-19 vaccination for the purpose of preventing a future SARS-COV2 invention or protecting a severe illness from SARS-COV2 infection. Typically, the patient or subject receiving treatment according to the method of this invention to enhance immunity of COVID vaccination or to reduce adverse events from COVID vaccination is not otherwise in need of treatment by the same therapeutic agents. For example, if a subject is receiving the probiotic or symbiotic composition(s) according to the claimed method, the subject is not suffering from any disease that is known to be treated by the same composition(s). Although a patient may be of any age, in some cases the patient is at least 20, 30, 40, 45, 50, 55, 60, 65, 70, 75, 80, or 85 years of age; in some cases, a patient may be between 20 and 30, 30 and 40, 40 and 45 years old, or between 50 and 65 years of age, or between 65 and 85 years of age. A “child” subject is one under the age of 18 years, e.g., about 2-5 or about 2-10, or about 5-17, 9 or 10-17, or 12-17 years old, including an “infant,” who is younger than about 12 months old, e.g., younger than about 10, 8, 6, 4, or 2 months old, whereas an “adult” subject is one who is 18 years or older.

The term “effective amount,” as used herein, refers to an amount that produces intended (e.g., therapeutic or prophylactic) effects for which a substance is administered. The effects include the prevention, correction, or inhibition of progression of the symptoms of a particular disease/condition and related complications to any detectable extent, e.g., incidence of disease, infection rate, one or more of the symptoms of a viral or bacterial infection and related disorder, or to achieve the promotion/enhancement of desirable effects and/or the prevention/reduction of undesirable adverse events (e.g., from a COVID-19 vaccine). The exact amount will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “about” when used in reference to a given value denotes a range encompassing ±10% of the value.

A “pharmaceutically acceptable” or “pharmacologically acceptable” excipient is a substance that is not biologically harmful or otherwise undesirable, i.e., the excipient may be administered to an individual along with a bioactive agent without causing any undesirable biological effects. Neither would the excipient interact in a deleterious manner with any of the components of the composition in which it is contained.

The term “excipient” refers to any essentially accessory substance that may be present in the finished dosage form of the composition of this invention. For example, the term “excipient” includes solvents, emulsifiers, vehicles, binders, disintegrants, fillers (diluents), lubricants, glidants (flow enhancers), compression aids, colors, sweeteners, preservatives, suspending/dispersing agents, film formers/coatings, flavors and printing inks.

The term “consisting essentially of,” when used in the context of describing a composition containing an active ingredient or multiple active ingredients, refer to the fact that the composition does not contain in detectable quantity other ingredients possessing any similar or relevant biological activity of the active ingredient(s) or capable of enhancing or suppressing the activity, whereas one or more inactive ingredients such as physiological or pharmaceutically acceptable excipients may be present in the composition. For example, a composition consisting essentially of active agents (for instance, one or more bacterial species listed in Tables 1-6 plus Bifidobacterium adolescentis, Roseburia faecis, Prevotella copri, Megamonas funiformis, and Megamonas hypermegale) effective for enhancing immunity and/or for reducing adverse effects upon COVID vaccination in a subject is a composition that does not contain any other agents that may have any detectable positive or negative effect on the same target process (e.g., enhancing immunity and/or reducing adverse effects from a COVID-19 vaccine) or that may increase or decrease to any measurable extent of the relevant parameters (e.g., incidence of future infection or severity of illness, including hospitalization and mortality) among the receiving subjects.

### DETAILED DESCRIPTION OF THE INVENTION

### I. Introduction

This invention describes specific bacterial species and combination thereof (e.g., beneficial bacterial species listed in Tables 1-6 plus Bifidobacterium adolescentis, Roseburia faecis, Prevotella copri, Megamonas funiformis, and Megamonas hypermegale) for enhancing efficacy and/or reducing potential adverse effects of COVID-19 vaccination in a subject, especially when the subject is a human adult or child not currently suffering from COVID-19 but at risk of exposure to SARS-COV2 and infection. The practical use of the invention includes development and manufacturing of commercial food products or health supplements, for example in the form of a powder, tablet, capsule, or liquid, which can be taken alone or added to food or beverages, as well as any other formulation suitable for use by fecal microbiota transplant (FMT), for various applications in connection with COVID-19 vaccination.

### II. Pharmaceutical Compositions and Administration

The present invention provides pharmaceutical compositions comprising an effective amount of one or more of the beneficial bacterial species listed in Tables 1-6 plus Bifidobacterium adolescentis, Roseburia faecis, Prevotella copri, Megamonas funiformis, and Megamonas hypermegale for enhancing efficacy of COVID-19 vaccination and/or for reducing potential adverse effects of COVID-19 vaccination in recipients who have just received or are soon to receive a COVID-19 vaccine, e.g., an inactivated vaccine or an RNA-based vaccine. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, Langer, Science 249: 1527-1533 (1990).

The pharmaceutical compositions of the present invention can be administered by various routes, e.g., systemic administration via oral ingestion or local delivery using a rectal suppository. The preferred route of administering the pharmaceutical compositions is oral administration at daily doses of about 10⁶ to about 10¹² CFU for the combination of all beneficial bacterial species listed in Tables 1-6 plus Bifidobacterium adolescentis, Roseburia faecis, Prevotella copri, Megamonas funiformis, and Megamonas hypermegale. When multiple bacterial species are administered to the subject, they may be administered either in one single composition or in multiple compositions. The appropriate dose may be administered in a single daily dose or as divided doses presented at appropriate intervals, for example as two, three, four, or more subdoses per day. The duration of administration may range from about 1 week to about 8 weeks, e.g., about 2 week to about 4 weeks, or for a longer time period (e.g., up to 6 months) as the relevant symptoms persist or as required to maintain an effective inhibition level (e.g., an sVNT inhibition of 60% or higher).

For preparing pharmaceutical compositions containing the beneficial bacteria identified in this disclosure, one or more inert and pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., any one or more of the beneficial bacterial species listed in Tables 1-6 plus Bifidobacterium adolescentis, Roseburia faecis, Prevotella copri, Megamonas funiformis, and Megamonas hypermegale. In tablets, the active ingredient is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 100% by weight of the active ingredient(s) (e.g., one or more of the beneficial bacterial species listed in Tables 1-6 plus Bifidobacterium adolescentis, Roseburia faecis, Prevotella copri, Megamonas funiformis, and Megamonas hypermegale). Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active ingredient(s), e.g., one or more of the beneficial bacterial species listed in Tables 1-6 plus Bifidobacterium adolescentis, Roseburia faecis, Prevotella copri, Megamonas funiformis, and Megamonas hypermegale, with encapsulating material as a carrier providing a capsule in which the active ingredient(s) (with or without other carriers) is surrounded by the carrier, such that the carrier is thus in association with the active ingredient(s). In a similar manner, sachets can also be included. Tablets, powders, sachets, and capsules can be used as solid dosage forms suitable for oral administration.

Liquid pharmaceutical compositions include, for example, solutions suitable for oral administration or local delivery, suspensions, and emulsions suitable for oral administration. Culture solutions of the active component (e.g., one or more of the beneficial bacterial species listed in Tables 1-6 plus Bifidobacterium adolescentis, Roseburia faecis, Prevotella copri, Megamonas funiformis, and Megamonas hypermegale) or sterile solutions of the active component in solvents comprising water, buffered water, saline, PBS, ethanol, or propylene glycol are examples of liquid or semi-liquid compositions suitable for oral administration or local delivery such as by rectal suppository. The compositions may contain pharmaceutically acceptable auxiliary substances as required to approximate physiological conditions, such as pH adjusting and buffering agents, tonicity adjusting agents, wetting agents, detergents, and the like. The resulting aqueous solutions may be packaged for use as is, or lyophilized, the lyophilized preparation being combined with a sterile aqueous carrier prior to administration.

Sterile solutions can be prepared by dissolving the active component (e.g., one or more of the beneficial bacterial species listed in Tables 1-6 plus Bifidobacterium adolescentis, Roseburia faecis, Prevotella copri, Megamonas funiformis, and Megamonas hypermegale) in the desired solvent system, and then passing the resulting solution through a membrane filter to sterilize it or, alternatively, by dissolving the sterile active component in a previously sterilized solvent under sterile conditions. Alternatively, sterile solution can be prepared by dissolving the heat-inactivated active component in the desired solvent system, or by first dissolving active component in the desired solvent system then apply heat to inactivate it. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

Single or multiple administrations of the compositions can be carried out with dose levels and pattern being selected by the treating physician. In any event, the pharmaceutical formulations should provide a quantity of an active agent sufficient to effectively enhance the efficacy of a vaccine and/or reduce or eliminate undesirable adverse effects of a vaccine.

### III. Additional Therapeutic Agents

Additional known therapeutic agent or agents may be used in combination with an active agent such as one or more of the beneficial bacterial species listed in Tables 1-6 plus Bifidobacterium adolescentis, Roseburia faecis, Prevotella copri, Megamonas funiformis, and Megamonas hypermegale in the practice of the present invention for the purpose of enhancing efficacy and/or reducing adverse effects of COVID-19 vaccination in a vaccine recipient who might be at risk of exposure to SARS-COV2 or infection of SARS-COV2. In such applications, one or more of the previously known effective prophylactic or therapeutic agents can be administered to patients concurrently with an effective amount of the active agent(s) either together in a single composition or separately in two or more different compositions.

For example, drugs and supplements that are known to be effective for use to prevent or treat COVID-19 include ivermectin, vitamin C, vitamin D, melatonin, quercetin, Zinc, hydroxychloroquine, fluvoxamine/fluoxetine, proxalutamide, doxycycline, and azithromycin. They may be used in combination with the active agents (such as any one or more of the beneficial bacterial species listed in Tables 1-6 plus Bifidobacterium adolescentis, Roseburia faecis, Prevotella copri, Megamonas funiformis, and Megamonas hypermegale) of the present invention to promote the efficacy of a COVID-19 vaccine and to reduce the potential adverse effects from a COVID-19 vaccine among patients at risk of exposure to SARS-COV2 and SARS-COV2 infection. In particular, the combination of Zinc, hydroxychloroquine, and azithromycin and the combination of ivermectin, fluvoxamine or fluoxetine, proxalutamide, doxycycline, vitamin C, vitamin D, melatonin, quercetin, and Zinc have demonstrated high efficacy in both COVID prophylaxis and therapy. Thus, these known drug/supplement or nutritheutical combinations can be used in the method of this invention along with the active components of one or more of the beneficial bacterial species listed in Tables 1-6 plus Bifidobacterium adolescentis, Roseburia faecis, Prevotella copri, Megamonas funiformis, and Megamonas hypermegale.

### IV. Kits

The invention also provides kits for enhancing efficacy of COVID-19 vaccination and/or for reducing potential adverse effects from COVID-19 vaccination to be applied to patients who are not currently infected with SARS-COV2 but are at risk of potential exposure and future infection in accordance with the method disclosed herein. The kits typically include a plurality of containers, each containing a composition comprising one or more of the bacterial species listed in Tables 1-6 plus Bifidobacterium adolescentis, Roseburia faecis, Prevotella copri, Megamonas funiformis, and Megamonas hypermegale. Further, additional agents or drugs that are known to be therapeutically effective for prevention and/or treatment of the disease, including for ameliorating the symptoms and reducing the severity of the disease, as well as for facilitating recovery from the disease (such as those described in the last section or otherwise known in the pertinent technical field) may be included in the kit. The plurality of containers of the kit each may contain a different active agent/drug or a distinct combination of two or more of the active agents or drugs. The kit may further include informational material providing instructions on how to dispense the pharmaceutical composition(s), including description of the type of patients who may be treated (e.g., human patients, adults or children, including those who may be overweight or obese, who are not currently diagnosed of COVID-19 but may be at risk of exposure to SARS-COV2 and may become infected), the dosage, frequency, and specific manner of administration, and the like.

### EXAMPLES

The following examples are provided by way of illustration only and not by way of limitation. Those of skill in the art will readily recognize a variety of non-critical parameters that could be changed or modified to yield essentially the same or similar results.

### BACKGROUND

The Coronavirus-2019 (COVID-19) global pandemic has affected over one million people worldwide. Vaccine has been developed to control the pandemic. However, some people developed adverse effects while some people showed inadequate antibody response towards the vaccine. The purpose of this invention is to pinpoint gut microbiota alterations and microorganism for reducing adverse effects and enhancing efficacy of vaccine for COVID-19. The practical use of the invention includes development and manufacturing of commercial food products or health supplements for example in the form of sachet, tablet, capsule, which can be taken alone or added to food or beverages.

### INTRODUCTION

Vaccine-induced immune responses are highly variable among different individuals and populations. Identifying the determinant factors to influence vaccine immunogenicity and vaccine are urgently needed. Increasing evidence from clinical studies and animal models now suggests that the composition and function of the gut microbiota are crucial factor modulating immune responses to vaccination. To address this, the present inventors conducted a prospective observational study to examine the gut microbiota determinants of immune responses and adverse events in adults who received either the inactivated virus vaccine (Corona Vac; Sinovac) or the mRNA vaccine (BNT162b2; BioNTech; Comirnaty). It was found that the percentage of inhibition and levels of RBD-specific IgG assessed by surrogate neutralization test (sVNT) and ELISA were lower in subjects with CoronaVac compared to those with BNT162b2. Using shotgun metagenomic analysis of fecal samples, Bifidobacterium adolescentis was persistently higher in those with high neutralizing antibodies to Corona Vac (as defined by achieving at least twice the 50% protection threshold for sVNT at one month post-second dose). They also had higher abundances of pathways related to carbohydrate metabolism and pathways that positively correlated with the abundance of Bifidobacterium adolescentis. Neutralizing antibodies among recipients of BNT162b2 vaccine showed a positive correlation with the total abundance of bacteria with flagellin and fimbriae including Roseburia faecis. The abundance of Prevotella copri and two Megamonas species were enriched in individuals with less adverse events following either of the vaccines indicating that these bacteria species may play an anti-inflammatory role in host immune response. The present study has identified gut microbiota determinants of immune responses and adverse events in adults who received Corona Vac and BNT162b2. Microbiota-targeted interventions have a potential not only to optimize immune responses to COVID-19 vaccines but also to minimize vaccine-related adverse events.

### DESCRIPTION OF STUDY

Vaccination elicits protective immune responses against the severe acute respiratory syndrome coronavirus 2 (SARS-COV-2) and provides hope for containing the coronavirus disease 2019 (COVID-19) pandemic. More than 6.6 billion doses of vaccine have been administrated worldwide1 and efficacy has been substantial in different countries²⁻⁴. Recently, observational studies found a steady decline of antibody levels among vaccinated individuals, which imply a growing risk of breakthrough infection over time^{5,6,7}. Factors that influence the immunogenicity and durability of vaccine is still not yet fully understood. Evidence from clinical or animal studies suggested that the composition and functions of the gut microbiota are crucial factors that modulate the immune responses of vaccination^{8,9}. Mucosal or systemic microbiota exposure shapes T and B cell repertoires that has an important implication for regulating responses to vaccination^{10,11}. Whether host microbiota composition can influence the responses of COVID-19 vaccines in humans has not been determined. The present inventors conducted a prospective observational study of human adults who received either the inactivated virus vaccine (Corona Vac; Sinovac) or the mRNA vaccine (BNT162b2; BioNTech; Comirnaty) to examine the gut microbiota determinants of immune responses and vaccine-related adverse events.

### Participant Demographics and Immune Responses

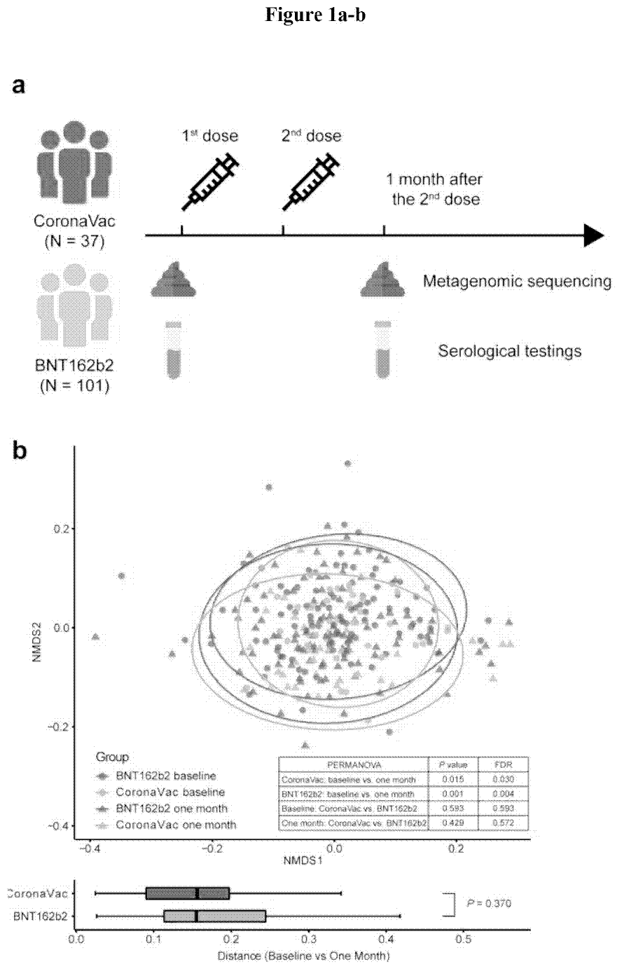

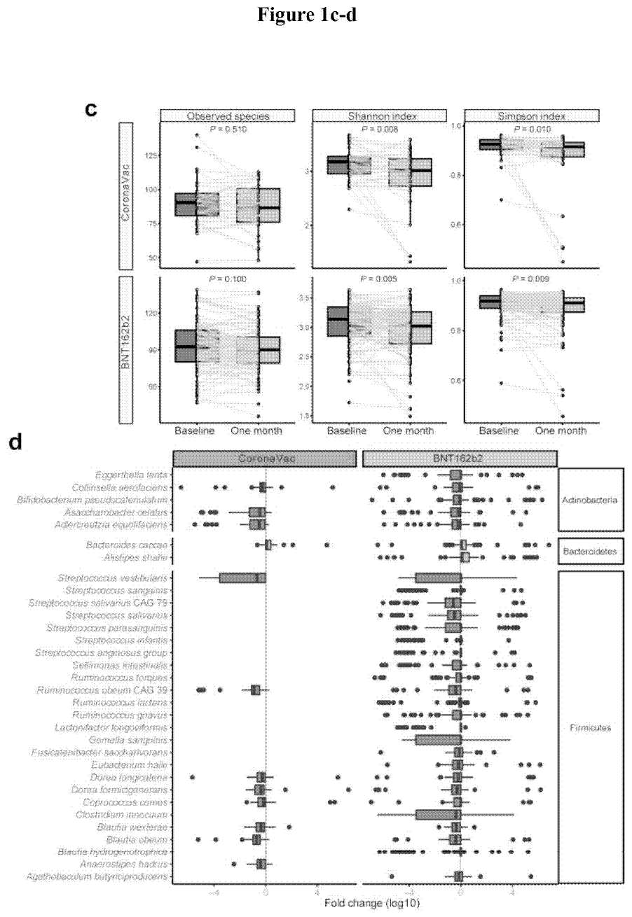

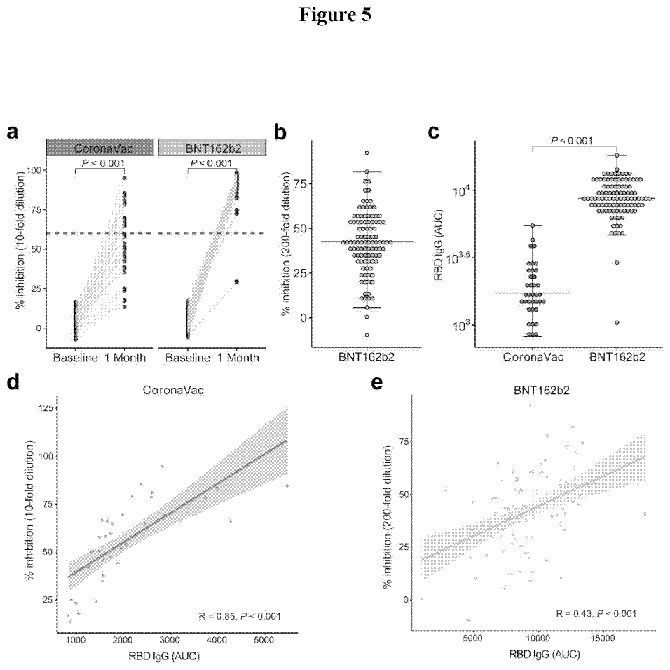

Between April 1 and Aug. 31, 2021, 138 adult volunteers who have received two doses of either the inactivated vaccines (Corona Vac; n=37) or the mRNA vaccine (BNT162b2; n=101) were recruited from The Chinese University of Hong Kong and The University of Hong Kong (FIG. 1a). Fecal and blood specimens were collected before vaccination (as baseline) and at one month after the second dose of vaccination from each participant. The participants ranged in age from 18-67 years (median=47 years, interquartile range; IQR: 31.2, 55.0) and that 32.6% was male. 38.4% was classified as overweight or obese (i.e. body mass index; BMI≥23) (Table 7). Compared to BNT162b2, Corona Vac vaccinees were older in age [55.0 (Corona Vac) vs. 42.0 (BNT162b2); P=0.003] and a higher proportion had hypertension [18.9% (CoronaVac) vs 6.9% (BNT162b2), P=0.055].The results of SARS-COV-2 surrogate Virus Neutralization Test (sVNT) and Anti-RBD IgG Test (ELISA) were negative in the plasma samples of all participants which were collected before vaccination. At one month after completion of two doses of the vaccines, Corona Vac vaccinees had significantly lower immune response against SARS-COV-2 compared with BNT162b2 vaccinees (sVNT: 57.6% vs. 95.2%, P<0.001; anti-RBD: 1725.0 vs.8696.0, P<0.001) (Table 7 and FIG. 5a-b). Moreover, the results of sVNT were negatively correlated with BMI in the Corona Vac group (BMI; Spearman's rho (R)=−0.385, P=0.018, Supplementary Table 1), and it was significant in both male and female (R=−0.817, P=0.007 and R=−0.403, P=0.033, respectively).

### Baseline Gut Microbiome Composition Predicts Immune Response of COVID-19 Vaccine

Shotgun metagenomic analysis was performed on stool samples to determine whether baseline gut microbiome composition was associated with the immune response to COVID-19 vaccines. In total, 272 stool samples were sequenced generating an average of 7.7 Gb (33.7M reads) per sample. It was observed that a significant change in the gut microbiome composition including shifts in beta diversity (FIG. 1b) and a decrease in alpha diversity (FIG. 1c) at one month after vaccination compared with baseline samples in both vaccine groups. These changes were not significantly different between the two vaccine groups. Baseline gut microbiome was significantly associated with several comorbidities, antibiotic use within 3 months prior to vaccination, regular exercise and recent symptoms of diarrhoea (Supplementary Table 2).

At the species-level, only the abundance of Bacteroides caccae was found to be increased in Corona Vac vaccinees whereas BNT162b2 vaccinees had increased abundances of both B. caccae and Alistipes shahii, one month after two doses of vaccination. On the other hand, a relative decline in abundances of common bacterial species including Adlercreutzia equolifaciens, Asaccharobacter celatus, Blautia obeum, Blautia wexlerae, Dorea formicigenerans, Dorea longicatena, Coprococcus comes, Streptococcus vestibularis, Collinsella aerofaciens, and Ruminococcus obeum CAG 39 (FIG. 1d) were observed in both vaccine groups. A significant decline in Actinobacteria and Firmicutes abundances could be explained by altered physiological functions and drastic inflammation during vaccine regimen¹². Importantly, none of the participants reported significant dietary changes during the study period. Among 72 randomly selected participants, it was found that no significant changes in details of dietary intakes recorded at baseline and at one month after the second dose of vaccine (P>0.05; Supplementary Table 3).

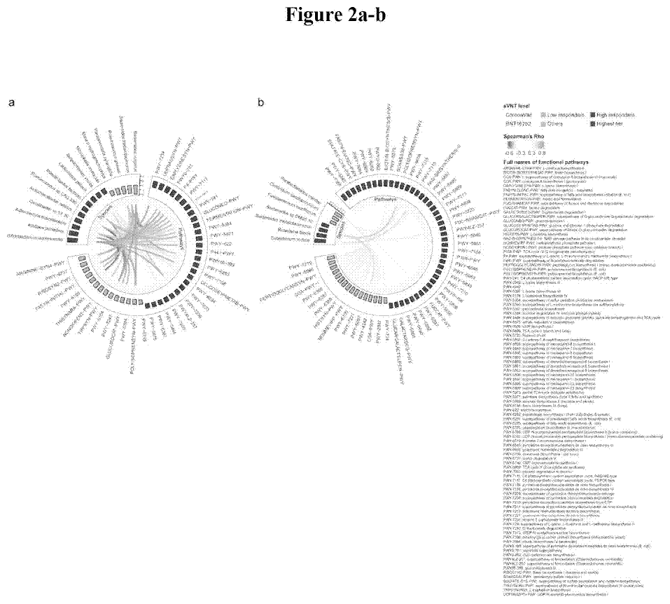

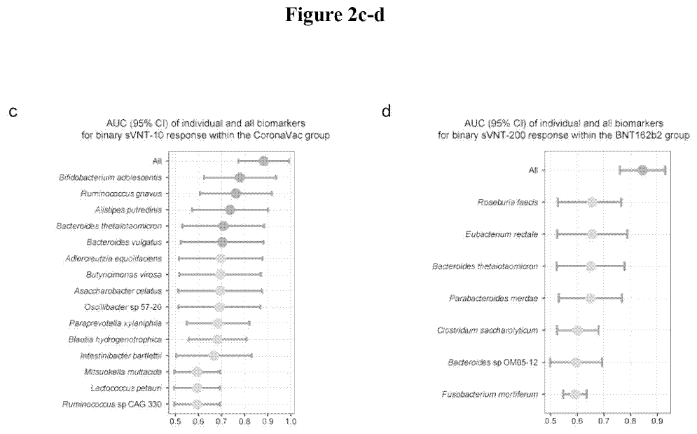

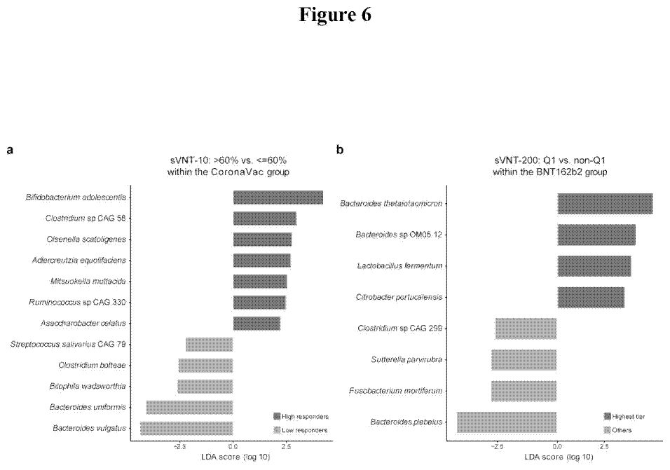

Consistent with previous findings, it was found that results of sVNT and RBD-specific ELISA were highly correlated (Spearman's rho (R)=0.85, P<0.001 in Corona Vac; R=0.48, P<0.001 in BNT162b2, FIG. 5c-d). Thus, the present inventors focused their analysis using results of sVNT^{13, 14}. Khoury and colleagues reported that 50% protection from neutralization was related to antibody levels that were 20% of convalescent antibody titers⁷. People with neutralizing antibody level lower than this level may be at risk of re-infection. Since there was waning of antibody from peak titres observed at one month after the second dose of vaccine, target titre was set and achieved at one month post-second dose of vaccine to be twice the 50% protection titre which corresponded to a sVNT inhibition of 60%¹⁴. Among Corona Vac vaccinees, 21 of 37 (56.8%) participants who showed sVNT lower than 60% (low-responders) had a distinct gut microbiome from those with sVNT higher than 60% (high-responders). In particular, a total of 15 bacteria species were identified, of which Bifidobacterium adolescentis was enriched in the high-responder group while Bacteroides vulgatus, Bacteroides thetaiotaomicron and Ruminococcus gnavus were more abundant in low-responders (FIG. 2a). At one month after the second dose of vaccination, seven species including B. adolescentis, A. equolifaciens and A. celatus were also found to be more abundant whereas B. vulgatus remained less abundant in high-responders (FIG. 6a). Using mixed effect modeling¹⁵, it was shown that B. adolescentis was persistently higher while B. vulgatus was persistently lower from baseline to one month after the second dose in the high-responder group (Supplementary Table 4). The functional pathways were further interrogated, and it was found that Corona Vac vaccinees with sVNT>60% had higher abundances of pathways related to carbohydrate metabolism and most of these pathways were positively correlated with the abundance of B. adolescentis (FIG. 2a). On the other hand, low-responders had relatively higher abundance of L-ornithine¹⁶ biosynthesis¹⁶ pathway which was positively correlated with abundances of B. vulgatus and B. thetaiotaomicron at baseline (FIG. 2a).

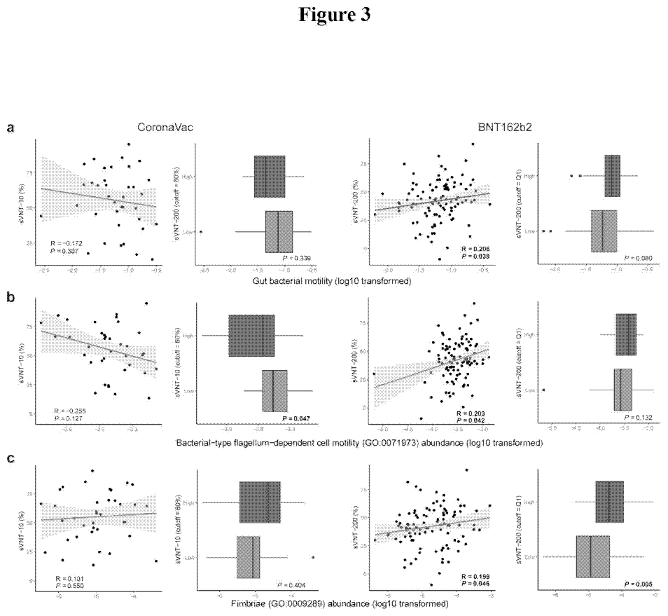

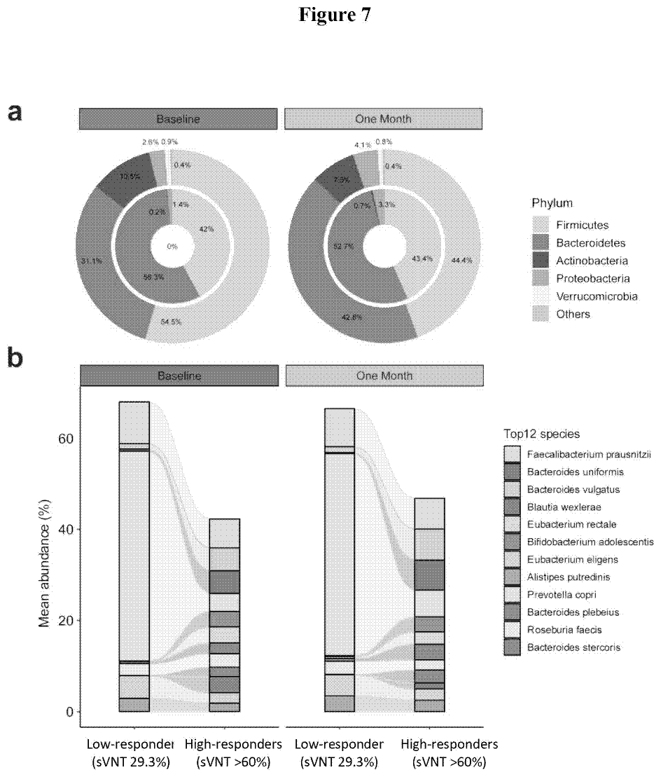

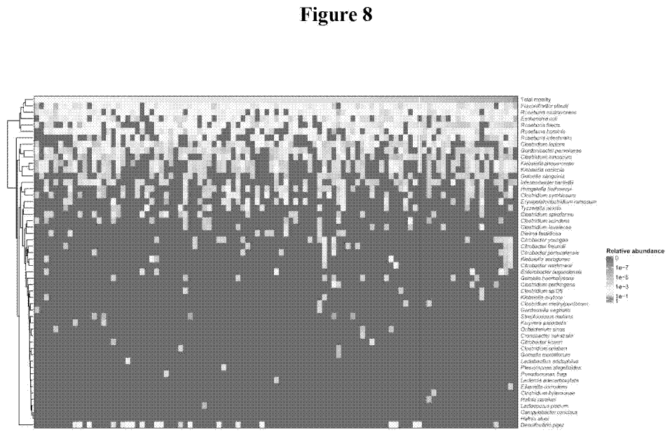

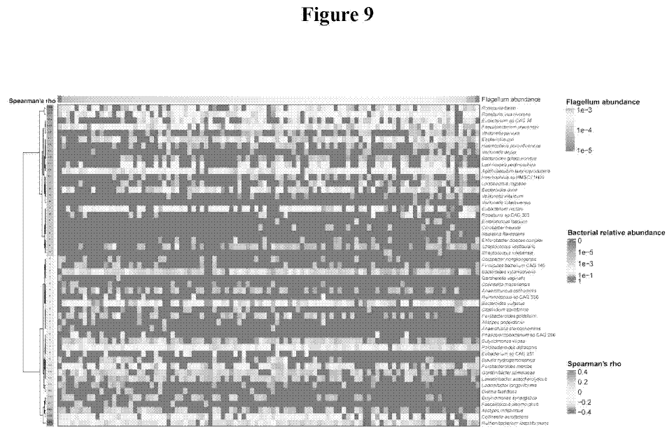

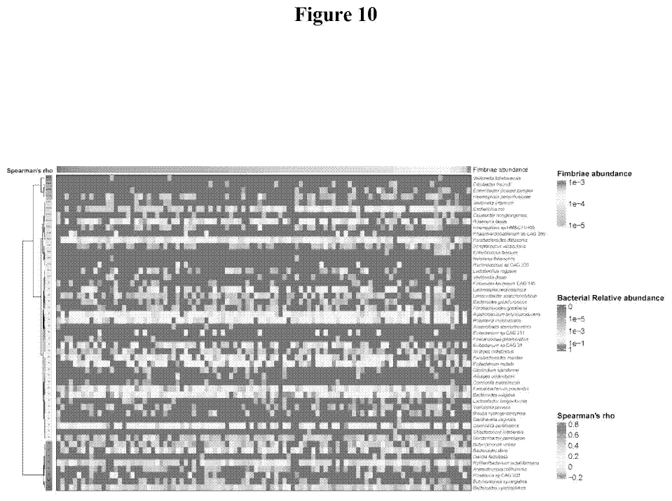

The sVNT kit has a ceiling of detection limit using the standard dilution¹⁷. Studies showed that most people who received the BNT162b2 vaccine reached this detection limit one month after the two doses of vaccination¹⁸. In this study, only one participant who received BNT162b2 vaccine had very low sVNT inhibition (29.3%) (FIG. 5a). The participant was overweight, had a history of kidney transplant and was on corticosteroids and antihypertensive therapy. Similar to Corona Vac low-responders, the gut microbiota of BNT162b2 low-responder had persistently low level of Actinobacteria particularly B. adolescentis (FIG. 7). To further differentiate response amongst the participants, sVNT was performed using plasma samples after 200 folds of dilution to further differentiate the neutralization level from the samples of BNT162b2 (FIG. 5b). We then defined the quartiles from the sVNT results of our BNT162b2 cohort. We found that four specific bacteria including Eubacterium rectale, Roseburia faecis and two Bacteroides species, B. thetaiotaomicron and Bacteroides sp OM05-12 were significantly increased in the highest-tier responders with top 25% of neutralizing antibody level (FIG. 2b). Interestingly, we found that a higher relative abundance of bacteria with flagellin were associated with a higher antibody response to BNT162b2 vaccine. R. faecis is one of the major contributors to gut bacterial motility, according to both bacterial phenotype database and Gene Ontology annotation (GO:0071973, FIGS. 8-9), which was positively correlated with the level of the results of sVNT in BNT162b2 vaccinees (FIG. 3a-b). Moreover, R. faecis and E. rectale are likely to express fimbriae (according to GO:0009289, FIG. 10), which was also positively correlated with the results of sVNT in BNT162b2 vaccinees (FIG. 3c). Among those bacterial biomarkers, the two Bacteroides species remained persistently enriched at one month after vaccination of BNT162b2 in the highest-tier responders (FIG. 6b). Notably, enriched pathways for biosynthesis of several menaquinols were found in the samples from the highest-tier responders which were collected before but not after vaccination. On the other hand, there was decreased abundance of pathways for adenosine¹⁹ ribonucleotide biosynthesis and for peptidoglycan biosynthesis (FIG. 2b).

The predictive power of the abovementioned bacterial species markers was further tested based on the area under the receiver operating characteristic curve (AUC) to each type of vaccine. The predictive power of B. adolescentis alone (AUC (95% CI): 0.780 (0.624-0.935) was higher than other bacteria species in predicting high-responders vs. low-responders to the inactivated vaccine, Corona Vac (FIG. 2c) but this was not significantly different from the AUC of combined bacteria species, 0.882 (0.773-0.992). For the mRNA vaccine, BNT162b2, the best predictive power was observed in the model using a combination of seven bacteria species, 0.845 (0.761-0.930) (FIG. 2d).

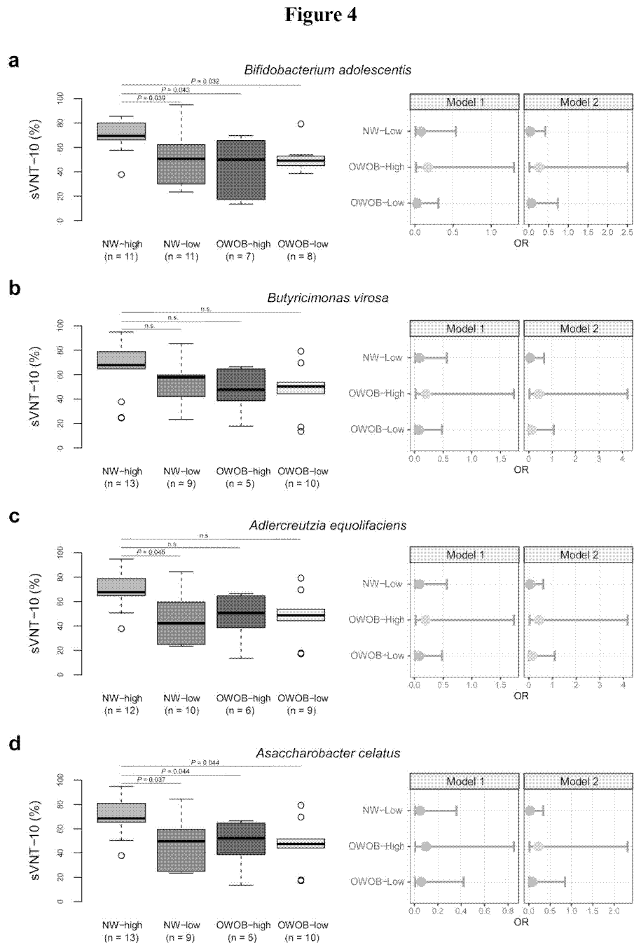

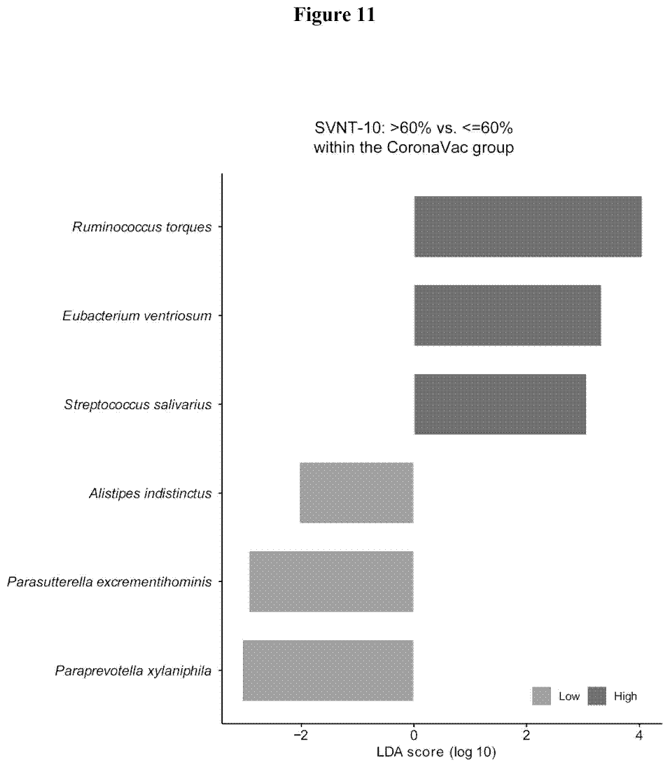

Gut microbiome is known to be influenced by host physiological status and lifestyle factors. Reciprocally, gut microbiome orchestrates the host immune system and modulates the responses to vaccines⁸. We found that the results of sVNT were correlated with BMI (Supplementary Table 1 and FIG. 4) and abundance of certain bacteria in the Corona Vac group. This observation prompted us to further investigate the potential role of weight as an effect modifier of bacteria-immune response relationship. Based on the comparison between strata of weight status and abundance of bacterial species markers, we found that the association of the four bacteria species with immune response were significantly influenced by body weight. The positive associations between the four bacterial biomarkers with immune response were compromised in overweight or obese (OWOB) people. These species include two short-chain fatty acid producers, B. adolescentis and Butyricimonas virosa, and A. equolifaciens and A. celatus (FIG. 4). However, compared with normal weight people with high abundances of B. adolescentis and A. celatus, the risk of being low-responders was not significant for overweight or obese people if they had high abundance of the same bacteria species (Model 2: adjusted OR 0.27, 95% CI 0.02, 2.51 and OR 0.43, 95% CI 0.04, 4.23, respectively). These results suggest that the beneficial effect of these bacteria on the immune responses to Corona Vac vaccine was attenuated in overweight or obese people. Therefore, we further identified specific bacteria biomarkers in high BMI population. LEfSe analysis showed enrichment of three bacteria species including Ruminococes torques, Eubacterium ventriosum and Streptococcus salivarius in Corona Vac high-responders who were overweight or obese (FIG. 11).

### Gut Microbiome Composition is Associated with Vaccination-Related Adverse Events

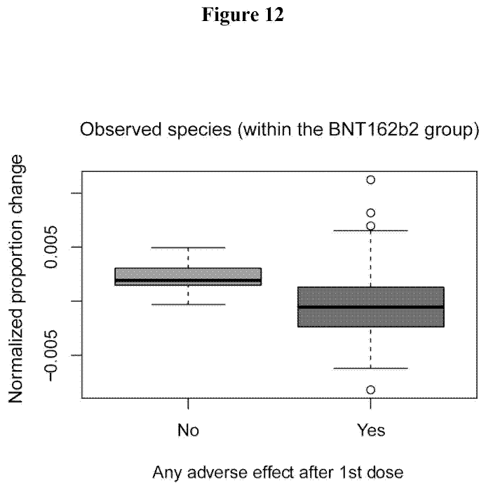

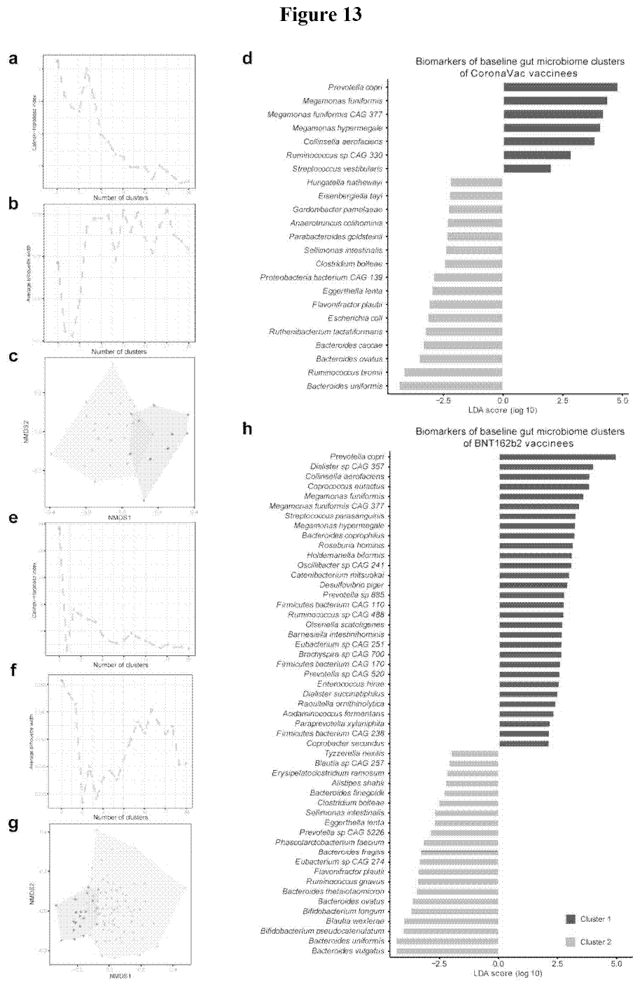

None of the participants had serious adverse events that lead to hospitalization. Consistent with reports in the literature, a greater proportion of BNT162b2 vaccinees reported adverse events than those with Corona Vac after both doses of vaccine²⁰. Significantly more participants developed injection site pain, fatigue, fever, myalgia, drowsiness, headache and chills after BNT162b2 compared with CoronaVac vaccines (Table 7 and Supplementary Table 5). We hypothesized that the gut microbiome composition may modulate adverse events caused by vaccination. Among the BNT162b2 group, participants who reported any adverse effect after first dose of vaccine had a significant decrease in observed bacteria species (P=0.011) (FIG. 12). To assess whether specific bacteria species were associated with vaccine-related adverse events, we applied an unsupervised clustering method (Partitioning around medoids (PAM) clustering)²¹, which optimally clustered the baseline gut microbiome composition of Corona Vac vaccinees into two distinct groups (FIG. 13a-c) with varying proportion of adverse events after both doses of vaccine (Supplementary Table 6). Consistent with previous studies including Asian populations^{22,23,24}, two distinct gut microbiota clusters can be distinguished primarily by levels of Bacteroides and Prevotella. The cluster associated with less adverse events to Corona Vac had a higher abundance of Prevotella copri and two Megamonas species (M. funiformis and M. hypermegale) in their gut microbiome (FIG. 13d). Similarly, the baseline gut microbiota cluster enriched by P. copri and the two Megamonas species is associated with less adverse events in BNT162b2 vaccinees (FIG. 13e-h), indicating that these species may play an anti-inflammatory role in both vaccine groups. Interestingly, symptoms of fatigue after the first dose of vaccination were associated with a higher level of inhibition in sVNT in BNT162b2 vaccinees but lower inhibition in Corona Vac vaccinees (Supplementary Tables 7 and 8).

### DISCUSSION

This is the first study to provide evidence that the gut microbiota plays an important role in modulating vaccine immunogenicity in humans. Differential bacteria species were found to be associated with higher vaccine response; the presence of the immunomodulatory bacteria, B. adolescentis, was associated with higher neutralizing antibodies to Corona Vac which suggests that specific bacteria may serve as adjuvant to potentially overcome the challenge of waning immunity and protection of inactivated vaccine. Interestingly, the abundance of P. copri and two Megamonas species were found to be more enriched in participants with less adverse events after inactivated and mRNA vaccines.

Increasing evidence from clinical studies⁹ and animal models^{25,26} suggest that the composition and function of the gut microbiota play a crucial role in modulating immune responses to vaccination. The mechanisms by which gut microbiota modulate immune responses to vaccination are not yet well understood. One potential mechanism is by providing natural adjuvants that enhance responses to vaccination⁸. Commonly used vaccine adjuvants directly or indirectly activate antigen-presenting cells such as dendritic cells (DCs) via pattern recognition receptors (PRRs) like TLRs or NOD-like receptors (NLRs), which also detect microbial molecules, including those produced by the microbiota²⁷. Flagellin and peptidoglycan produced by the microbiota, as the natural adjuvants, could be sensed by PRRs⁸. For example, TLR5-mediated sensing of flagellin produced by the gut microbiota has been shown to be required for optimal antibody responses to non-adjuvanted influenza vaccine²⁶. Moreover, the adhesin portion of bacterial fimbriae can induce the innate immune system via TLR4²⁸, which is one of the immune activator proteins that has been proposed as an effective adjuvant for mRNA vaccines²⁹. In consistent, it was found that a higher relative abundance of bacteria (E. rectale, R. faecis) with flagellin and fimbrae were associated with a higher antibody response to mRNA vaccine. Short-chain fatty acids (SCFAs) produced by the microbiota also enhanced B cell metabolism and gene expression to support optimal homeostatic and pathogen-specific antibody responses³⁰. Being butyrate-producers, the enrichment of E. rectale and R. faecis, would be the explanation for elevated immunogenicity in highest-tier BNT162b2 responders. Therefore, these gut microbiota species may play a beneficial role in vaccine immunogenicity as adjuvants through immunomodulatory TLR agonists. Given that BNT162b2 COVID-19 vaccine effectiveness drops after 6 months³¹, whether microbiota-produced flagellin/fimbirae or SCFAs contributes to sustaining the long-term immunization to non-adjuvanted BNT162b2 vaccine is currently unknown but this potential mechanism by which the gut microbiota could influence vaccine responses is worthy of further investigation.

The potential role played by gut microorganisms in immunity boosting on COVID-19 vaccines could allow the use of a microbiome-based prediction model to stratify individuals with optimal response to vaccines or not. In light of previous reports that B. adolescentis³², E. retale, R. faecis³³ might have immunomodulatory properties to alter innate immune responses to vaccines, it was found that B. adolescentis was enriched in Corona Vac high-responders (sVNT>60%) while E. retale, R. faecis, B. theaiotaomicron and Bacteroides. sp OM05-12 were significantly increased in BNT162b2 highest-tier responders. Moreover, a relatively low level of B. adolescentis was identified in a single BNT162b2 vaccinee with very low level of neutralizing antibodies. Previous studies in infant population suggested that the abundance of Bifidobacteria were significantly associated with CD4 T cell responses and increased antibody responses to several vaccines^{34,35}. A recent study also reported that vaccine-induced T cell responses showed broad cross-reactivity against SARS-COV-2 variants³⁶. Thus, gut microbiota associated T cell responses would benefit not only vaccine immunogenicity but also cross-protection against multiple variants. In association with the abundance of B. adolescentis, we also observed enriched carbohydrate metabolic pathways in Corona Vac high-responders. Carbohydrates play crucial roles in the immune system function and the stimulation of the immune response, thus considering them as promising vaccine adjuvants³⁷. Our results indicate that B. adolescentis can indirectly induce carbohydrate-based immunopotentiating effects. These data indicate that vaccinees who have higher abundance of these bacteria may have a superior immune response and potentially a higher protection.

Obesity is often associated with an adverse impact on the immune system, it may thus modulate the effect of vaccines on antibody production. A recent study reported an inverse correlation between the titre of antibody against the SARS-COV-2 spike protein and BMI in men who received BNT162b2 vaccine³⁸. However, there is no report on the role of BMI in immunogenicity, in the durability of neutralizing responses, and in protection in Corona Vac vaccinees. Herein, we observed that immune response based on percent inhibition in sVNT was correlated with BMI and the abundance of certain bacteria (B. adolescentsi, B. virosa, A. equolifaciens and A. celatus) in CoronaVac vaccinees. These results suggest that the beneficial effect of these bacteria on immune responses to Corona Vac vaccine was modified by body weight. Importantly, we identified specific probiotics in high-responders (R. torques, E. ventriosum and S. salivarius) that might be more beneficial as targeted intervention in overweight and obese subjects.

In line with a previous study²⁰, the present inventors observed a greater proportion of BNT162b2 vaccinees experienced more adverse events than Corona Vac vaccinees, including injection site pain/burn, fever, and myalgia. Interestingly, the gut microbiota cluster with a higher abundance of P. copri and two Megamonas species was related to less adverse events in both vaccine groups, perhaps through their anti-inflammatory functions. Higher prevalence of P. copri has been consistently reported in non-westernized populations³⁹. It was shown in a rat model to enhance farnesoid X receptor (FXR) signalling⁴⁰, which has anti-inflammatory effect⁴¹ via modulating bile acid metabolism. Amongst the two Megamonas species, M. funiformis could ferment glucose into acetate and propionate^{42,43} which are beneficial for immune homeostasis; whereas M. hypermegale are important for the balance between regulatory T cell and type 17 helper T cells (Th17)⁴⁴.

This study demonstrates that human gut microbiota are highly associated with immunogenicity and adverse events of COVID-19 vaccines. Several gut bacterial species can predict immunogenicity to both inactivated and mRNA COVID-19 vaccines. These novel findings can help facilitate microbiota-targeted interventions to optimize immune response to vaccination and potentially enhance durability of protection.

### SUMMARY OF FINDINGS

### Bacterial Species for Enhancing SARS-COV-2 Antibody Response in Subjects in Need of Vaccination of an Inactivated Vaccine against COVID-19

Following vaccination using an inactivated COVID-19 vaccine, such as SinoVac-CoronaVac, not everyone developed adequate neutralizing antibody, which is an indicator of protective immunity against SARS-COV-2. High-responders are defined as those with sVNT inhibition of 60% inhibition or higher. In one cohort, 56.8% of subjects are low-responders to vaccine (as defined by having sVNT inhibition of lower than 60%). These subjects are characterized by a distinct microbiome from high-responders (having sVNT inhibition of 60% or higher). In particular, it was discovered that high-responders tend to have higher level of the bacterial species listed in Table 1, especially B. adolescentis which has shown better predicting power for high-responders vs. low-responders to the inactivated vaccine, Corona Vac than other bacteria in Table 1. This discovery enables different methods to enhance production of neutralizing antibody against SARS-COV-2, by adjusting or modulating the level of these bacterial in the GI tract of a subject in need of vaccination to deliver to the subjects' GI tract an effective amount of one or more of the bacterial species shown in Table 1, especially B. adolescentis.

TABLE 1
Bacterial species for enhancing antibody
response to inactivated COVID-19 vaccine

| | | Relative Abundance in Responders | | |
|---|---|---|---|---|
| | | | Lower | Upper |
| Species | NCBI:txid | Median | Quartile | Quartile |
| Bifidobacterium | 1680 | 3.86% | 1.32% | 8.39% |
| adolescentis | | | | |
| Alistipes putredinis | 28117 | 3.14% | 0.60% | 5.03% |
| Adlercreutzia | 446660 | 0.36% | 0.25% | 0.59% |
| equolifaciens | | | | |
| Oscillibacter sp. 57_20 | 1897011 | 0.61% | 0.16% | 0.84% |
| Asaccharobacter celatus | 394340 | 0.12% | 0.06% | 0.20% |
| Ruminococcus | 1262954 | 0.00% | 0.00% | 0.00% |
| sp. CAG: 330 | | | | |
| Intestinibacter bartlettii | 261299 | 0.01% | 0.00% | 0.03% |
| Lactococcus petauri | 1940789 | 0.00% | 0.00% | 0.00% |
| Mitsuokella multacida | 52226 | 0.00% | 0.00% | 0.00% |
| Butyricimonas virosa | 544645 | 0.04% | 0.00% | 0.10% |

In a sub-group of subjects who are obese or overweight, low responders tend to have lower relative abundance of bacterial species listed in Table 2, FIG. 11. Thus, to enhance production of neutralizing antibody against SARS-COV-2, the level of these bacterial species in the GI tract of an obese or overweight subject in need of vaccination should be adjusted or modulated by delivering to the subjects' GI tract an effective amount of one or more of the bacterial species shown in Table 2, independently or in addition to the bacterial species shown in Table 1.

TABLE 2
Bacterial species for enhancing antibody response to inactivated
COVID-19 vaccine in overweight and obese subjects

| | | Relative Abundance in Responders | | |
|---|---|---|---|---|
| | | | Lower | Upper |
| Species | NCBI:txid | Median | Quartile | Quartile |
| Ruminococcus torques | 33039 | 0.82% | 0.13% | 1.80% |
| Eubacterium ventriosum | 39496 | 0.13% | 0.01% | 0.17% |
| Streptococcus salivarius | 1304 | 0.06% | 0.02% | 0.46% |

### Bacterial Species for Enhancing SARS-COV-2 Antibody Response in Subjects in Need of Vaccination of an mRNA Vaccine against COVID-19

Antibody response rate to mRNA vaccine such as BioNTech vaccine is generally much higher than that of inactivated vaccine. However, by further enhancing the antibody response, there is a potential to reduce the number of doses of vaccine while maintaining the same level of efficacy. Similar to Corona Vac low-responders, the gut microbiota of BioNTech low-responder (sVNT<60%) had persistently very low level of Actinobacteria particularly Bifidobacterium adolescentis (FIG. 7). It was discovered that subjects in the highest tier also referred to as highest-tier responders (>25% of the study population) of neutralizing antibody level are characterized by a higher level of bacterial species listed in Table 3, FIG. 2b. Therefore, to enhance production of neutralizing antibody against SARS-COV-2, the level of these bacterial species in the GI tract of a subject in need of vaccination of an mRNA COVID-19 vaccine, should be adjusted or modulated by delivering to the subjects' GI tract an effective amount of one or more of the bacterial species shown in Table 3, FIG. 2b. Among these species, Bacteroides thetaiotaomicron and Bacteroides sp. OM05-12 species was persistently enriched in the highest-tier responders at one month (FIG. 6b and Supplementary Table 4). Notably, enriched pathways for biosynthesis of several menaquinols were found from the samples of the highest-tier responders which were collected before but not after vaccination (FIG. 2b). Therefore, supplementation of menaquinols before vaccination may be beneficial to enhance vaccine response for mRNA vaccine.

TABLE 3
Bacterial species for enhancing antibody
response to mRNA COVID-19 vaccine

| | | Relative Abundance in | | |
|---|---|---|---|---|
| | | High-tier Responders | | |
| | | | Lower | Upper |
| Species | NCBI:txid | Median | Quartile | Quartile |
| Eubacterium rectale | 39491 | 1.20% | 0.00% | 4.09% |
| Roseburia faecis | 301302 | 0.95% | 0.01% | 4.34% |
| Bacteroides | 818 | 0.25% | 0.10% | 0.78% |
| thetaiotaomicron | | | | |
| Bacteroides sp. OM05-12 | 2292283 | 0.00% | 0.00% | 0.00% |

In addition, it was found that higher relative abundance of bacteria with flagellin are associated with better antibody response to mRNA COVID-19 vaccine (FIG. 3a, motile gut microbiome). These bacteria are listed in Table 4. In particular, Roseburia faecis is one of the major contributors to gut bacterial motility (FIGS. 8 and 9), which was also positively correlated with the level of the percentage inhibition in sVNT within this vaccine group (FIG. 2d).

TABLE 4
Bacterial species with flagellin for enhancing
antibody response to mRNA COVID-19 vaccine

| | | Relative Abundance in | | |
|---|---|---|---|---|
| | | Higher-tier Responders | | |
| | | | Lower | Upper |
| Species | NCBI:txid | Median | Quartile | Quartile |
| Flavonifractor plautii | 292800 | 0.24% | 0.08% | 0.67% |
| Roseburia inulinivorans | 360807 | 1.02% | 0.17% | 2.39% |
| Roseburia faecis | 301302 | 0.92% | 0.01% | 4.46% |
| Roseburia hominis | 301301 | 0.17% | 0.01% | 0.49% |
| Escherichia coli | 562 | 0.09% | 0.01% | 0.50% |
| Gordonibacter | 471189 | 0.02% | 0.00% | 0.08% |
| pamelaeae | | | | |
| Clostridium innocuum | 1522 | 0.00% | 0.00% | 0.04% |
| Roseburia intestinalis | 166486 | 0.03% | 0.00% | 1.08% |
| Clostridium leptum | 1535 | 0.01% | 0.00% | 0.08% |

In a sub-group of subjects who are obese or overweight, non-responders to mRNA COVID-19 vaccine had lower relative abundance of bacterial species listed in Table 5. Thus, to enhance production of neutralizing antibody against SARS-COV-2, the level of these bacterial species in the GI tract of an obese or overweight subject in need of mRNA COVID-19 vaccination should be adjusted or modulated by delivering to the subjects' GI tract an effective amount of one or more of the bacterial species shown in Table 5, independently or in addition to the bacterial species shown in Tables 3 and 4.

TABLE 5
Bacterial species for enhancing antibody response to mRNA
COVID-19 vaccine in overweight and obese subjects

| | | Relative Abundance in | | |
|---|---|---|---|---|
| | | Higher-tier Responders | | |
| | | | Lower | Upper |
| Species | NCBI:txid | Median | Quartile | Quartile |
| Bacteroides | 821 | 2.16% | 0.49% | 6.05% |
| vulgatus | | | | |
| Eubacterium | 39491 | 1.40% | 0.12% | 4.18% |
| rectale | | | | |
| Roseburia | 301302 | 0.70% | 0.00% | 3.93% |
| faecis | | | | |

### Bacterial Species for Reducing Adverse Effects Following Vaccination of an Inactivated Vaccine against COVID-19

In the cohort 62.2% and 67.6% of subjects experienced one or more adverse effects (listed in Table 7) after the first and second dose of vaccination of Sinovac-Corona Vac respectively. Subjects experiencing adverse effects tend to have lower relative abundance of bacterial species listed in Table 6. Thus, to reduce adverse effects following vaccination, the level of these bacterial species in the GI tract of an obese or overweight subject in need of vaccination should be adjusted or modulated by delivering to the subjects' GI tract an effective amount of one or more of the bacterial species shown in Table 6.

TABLE 6
Bacterial species for reducing adverse effects
following inactivated vaccine against COVID-19

| | | Relative Abundance in Responders | | |
|---|---|---|---|---|
| | | | Lower | Upper |
| Species | NCBI:txid | Median | Quartile | Quartile |
| Asaccharobacter celatus | 394340 | 0.07% | 0.00% | 0.14% |
| Butyricimonas virosa | 544645 | 0.01% | 0.00% | 0.07% |
| Butyricimonas | 544644 | 0.00% | 0.00% | 0.00% |
| synergistica | | | | |
| Oxalobacter formigenes | 847 | 0.00% | 0.00% | 0.00% |
| Clostridium | 1262785 | 0.00% | 0.00% | 0.00% |
| sp. CAG: 253 | | | | |
| Clostridium | 1262792 | 0.00% | 0.00% | 0.00% |
| sp. CAG: 299 | | | | |
| Enterobacter cloacae | 354276 | 0.00% | 0.00% | 0.00% |
| complex | | | | |
| Lactococcus garvieae | 1363 | 0.00% | 0.00% | 0.00% |

### Prevotella copri, Megamonas funiformis, and Megamonas hypermegale for Reducing Adverse Effects Following Vaccination of mRNA Vaccine and Inactivated Vaccine against COVID-19

To assess whether specific bacteria species were associated with vaccine-related adverse events, we applied an unsupervised clustering method (Partitioning around medoids (PAM) clustering)²¹, which optimally clustered the baseline gut microbiome composition of Corona Vac vaccinees into two distinct groups (FIG. 13a-c) with varying proportion of adverse events after both doses of vaccine (Supplementary Table 6). The cluster associated with less adverse events to Corona Vac had a higher abundance of Prevotella copri and two Megamonas species (M. funiformis and M. hypermegale) in their gut microbiome (FIG. 13d). Similarly, the baseline gut microbiota cluster enriched by P. copri and the two Megamonas species is associated with less adverse events in BNT162b2 vaccinees (FIG. 13e-h), indicating that these species may play an anti-inflammatory role in both vaccine groups. Thus, to reduce adverse effects following vaccination of inactivated or mRNA COVID-19 vaccine, the level of these bacterial species in the GI tract of a subject in need of vaccination should be adjusted or modulated by delivering to the subjects' GI tract an effective amount of one or more of the bacterial species selected from Prevotella copri (NCBI: txid 165179), Megamonas funiformis (NCBI: txid 437897), Megamonas hypermegale (NCBI: txid 158847).

### Administration of Bacterial Species for Enhancing Antibody Response and Reducing Adverse Effects Following Vaccination against COVID-19

The bacterial species listed in Tables 1-6 can be obtained from a bacterial culture in a sufficient quantity and then formulated into a suitable composition, to be introduced into the subject by oral, nasal, or rectal administration. The amount of each of the bacteria in the composition is about 1×10⁶-1×10¹² CFU. Such composition can be taken for about 4 weeks prior to vaccination and continue for 6 months after vaccination. Ideally, the relative abundance should reach the median relative abundance (cut-off value), or within the range of lower and upper quartile listed in Tables 1-6, at the time the subject receives the first dose of vaccination. Some of these species, although only present in low relative abundance (<0.005%), still play an important role in enhancing antibody response or reducing adverse effects. For these species with a median relative abundance <0.005%, a detectable level e.g. at >0.005%. could be used as cut-off value.

### METHODS

### Study Cohorts

Participants were volunteers receiving either the mRNA vaccine (BNT162b2; N=101) or the inactivated virus vaccine (Corona Vac; N=37) against severe acute respiratory syndrome coronavirus 2 (SARS-COV-2) who were recruited for serial blood and stool donations at the Chinese University of Hong Kong (CUHK) Medical Centre or staff clinic of the Prince of Wales Hospital or at the University of Hong Kong (HKU)-School of Public Health or the Queen Mary Hospital, Hong Kong between April 1 and Aug. 31, 2021. Eligible participants were healthy adults aged 18 or above with no history of infection with SARS-COV-2, as determined by clinical history receiving either the of the currently available vaccine in Hong Kong, mRNA vaccine (BNT162b2) or the inactivated virus vaccine (Corona Vac), according to current dosing and interval guidelines. Exclusion criteria included incomplete vaccination status, presence of clinical signs and symptoms suggestive of acute infection with a positive reverse transcription polymerase chain reaction (RT-PCR) results for SARS-COV-2 in saliva, or a positive COVID-19 serology. Participants presented for blood and stool sample collection at baseline before vaccination and at one month after completing second dose of vaccines and were asked of possible adverse events, comorbidities and COVID-19 history. One stool sample and 10 ml of heparinized blood were collected from the participants at baseline and a one month after two doses of vaccine. Clinical data collection and management were carried out using REDCap (a secured web-based Research Electronic Data capturing system). All participants provided written informed consent before participation in the study and the study was conducted in accordance with Good Clinical Practice. The study was approved by The Joint Chinese University of Hong Kong—New Territories East Cluster Clinical Research Ethics Committee (The Joint CUHK-NTEC CREC) (2021.260) and HKU/HA HKW Institutional Review Board (UW 21-203). The study was conducted in accordance with the Declaration of Helsinki (1975) and Good Clinical Practice. For detailed participant characteristics see Table 7 and Supplementary Table 2.

### Clinical Data Collection

A standardised and structured questionnaire was used to capture basic demographics and adverse events after both doses of vaccine. Demographic data included age, gender, weight, height, comorbidities (hypertension, diabetes mellitus, allergy, diarrhoea, any other comorbidities), medication (antibiotics, hormone, immunomodulator), probiotics, vaccination in the past year, diet, alcohol intake (within 2 weeks prior to the 1^{ST} vaccination), regular exercise (strenuous/moderate). Body Mass Index was calculated and overweight or obese was determined according to the Asian-specific cut-off point of BMI≥23. Participants were asked to complete both questionnaires with assistance from a trained research staff. Adverse events questionnaire are summarized in the Supplementary Tables 7 and 8.

### Serological Tests

SARS-COV-2 surrogate virus neutralization test (sVNT) and RBD IgG ELISA were used to assess the antibody level in plasma collected before and one month after the 2^{nd} dose of vaccination. sVNT kits were obtained from GenScript, Inc., NJ, USA, and the tests were carried out according to the manufacturer's instructions. In brief, 10 μl of plasma was diluted at 1:10 and mixed with an equal volume of horseradish peroxidase (HRP) conjugated to SARS-COV-2 spike receptor binding domain (RBD) (6 ng). After incubation for 30 min at 37° C., a 100-μl volume of each mixture was added to each well on the microtiter plate coated with ACE-2 receptor. The plate was sealed and incubated at room temperature for 15 min at 37° C. The plate was then washed with wash solution and 100 μl of 3,3′,5,5′-tetramethylbenzidine (TMB) solution was added to each well and incubated in the dark at room temperature for 15 min. The reaction was stopped by addition of 50 μl of Stop Solution to each well and the absorbance read at 450 nm in an ELISA microplate reader. The assay validity was based on values representing optical density at 450 nm (OD450) for positive and negative results falling within the range of recommended values. On the basis of the assumption that the positive and negative controls gave the recommended OD450 values, percentage of inhibition of each plasma was calculated as follows: Inhibition (%)=(1−sample OD value/negative-control OD value)×100. Inhibition values of >20% are regarded as positive^{45.46}.

SARS-COV-2 spike receptor binding domain ELISA was carried out as previously described (6). ELISA 96-well plates (Nunc MaxiSorp; Thermo Fisher Scientific) were coated overnight with 100 ng per well of the purified recombinant RBD protein in phosphate-buffered saline (PBS) buffer. The plates were then blocked with 100 μl of ChonBlock blocking/sample dilution ELISA buffer (Chondrex Inc., Redmond, WA, USA) and incubated at room temperature for 2 h. Each plasma sample was tested from the dilution of 1:100 to 1:12800 in ChonBlock blocking/sample dilution ELISA buffer and added to the ELISA wells of each plate for 2 h of incubation at 37° C. After extensive washing with PBS containing 0.1% Tween 20, horseradish peroxidase (HRP)-conjugated goat anti-human IgG (GE Healthcare) (1:5,000) was added for 1 h at 37° C. The ELISA plates were then washed five times with PBS containing 0.1% Tween 20. Subsequently, 100 μl of HRP substrate (Ncm TMB One; New Cell and Molecular Biotech Co. Ltd., Suzhou, China) was added into each well. After 15 min of incubation, the reaction was stopped by adding 50 μl of 2 M H₂SO₄ solution and analyzed on an absorbance microplate reader at 450-nm wavelength. The validation and optical density cutoff for a positive result were as described in the previous publication^{45,46}. The Area under curve of each sample was calculated by GraphPad software.

### Stool DNA Extraction and Metagenomic Sequencing

The fecal DNA was extracted from the pellet using Maxwell RSC PureFood GMO and Authentication Kit (Promega, Madison, WI) following the manufacturer's instructions. Briefly, the fecal pellet was added to 1 mL of CTAB buffer and vortexed for 30 seconds, then the sample was heated at 95° C. for 5 minutes. After that, the samples were vortexed thoroughly with beads at maximum speed for 15 minutes. Then, 40 μL of proteinase K and 20 μL. of RNase A was added to the sample and the mixture was incubated at 70° C. for 10 minutes. The supernatant was then obtained by centrifuging at 13,000 g for 5 minutes and was added into the Maxwell RSC machine for DNA extraction. Extracted DNA was subject to DNA libraries construction, completed through the processes of end repairing, adding A to tails, purification and PCR amplification, using Nextera DNA Flex Library Preparation kit (Illumina, San Diego, CA). Libraries were subsequently sequenced on our in-house sequencer Illumina NovaSeq 6000 (250 base pairs paired-end) at the Microbiota I-Center (MagIC), Hong Kong, China. High quality sequence data generated for this study are available in the Sequence Read Archive under BioProject accession PRJNA650244.

### Sequence Data Processing and Analysis

Raw reads were quality filtered and trimmed using KneadData v0.10.0 with Trimmomatic v0.39 to remove adaptor and low-quality sequences (Parametersetting: “MINLEN:50 ILLUMINACLIP:TruSeq3-PE.fa:2:40:15 SLIDINGWINDOW:4:20”) and with Bowtie2 (Parameter settings: “—very-sensitive—dovetail”) to remove human host DNA by mapping reads onto human reference genome GRCh38. We acquired 2096.78 Gb high-quality pairedend reads for the 272 samples with an average of 7.71 Gb per sample. Following this, microbiota taxonomic compositions and functional potentials (including functional pathways and Gene Ontologies) were inferred from quality-filtered reads using MetaPhlAn (v3.0) and HUMAnN (v3.0), respectively, with default settings. Beta diversity (between-sample diversity) was calculated as Jensen-Shannon Divergence (JSD) index by phyloseq and vegan packages and visualized by non-metric multidimensional scaling (NMDS). Alpha diversity (within-sample diversity) indices, including observed species, Shannon and Simpson index, were calculated on the basis of the species profile for each sample. Gut microbial motility was calculated based on species relative abundance and motility phenotype (GOLD database (v202109) and IJSEM database), per Guittar et al⁴⁷.

Statistical Analysis

The primary analysis is to compare the relationship between microbiome profile and immune response in subjects with who have received different types of COVID-19 vaccines in Hong Kong. Baseline characteristics and adverse events after first and second dose of each vaccination group were compared using Fisher's exact test for the categorical variables and Wilcoxon rank-sum test. Pairwise multilevel comparisons among baseline and one-month samples of BNT162b2 and Corona Vac vaccinees were carried out on the JSD distance matrix using pairwise Adonis test. Associations between gut microbial community composition and patients' characteristics were assessed using permutational multivariate analysis of variance (PERMANOVA). Unsupervised clustering were conducted using the partitioning around medoids (PAM) clustering method based on the JSD distance matrix, and the number of clusters were determined according to the Calinski-Harabasz index, Silhouette coefficient and sample sizes. Pairwise Wilcoxon rank-sum tests were performed to compare the a-diversity of baseline and one-month samples within each vaccine groups. Differentially abundant species between groups/clusters were identified using the linear discriminant analysis effect size (LEfSe v1.1.01). Correlations between continuous variables, including immune responses (sVNT %, RBD-specific IgG level, species abundance and function abundance) were analyzed using Spearman's correlation tests, while the immune response differences amongst other binary/categorical variables to were tested by Wilcoxon rank-sum tests. GLM for binominal outcomes (high-responders versus low-responders or highest-tier responders vs others) with receiver operating characteristic (ROC) curve was applied to determine the prediction value of the identified biomarkers. Generalized linear models were also constructed to investigate modification effects while adjusting for potential confounders identified in univariable analysis. Mixed effect models were built to identify persistently differentially abundant species, using lme or lme.zig (of the NBZIMM package) that was optimized for zero-inflated microbiome data¹⁵, where appropriate. P values less than 0.05 were considered statistically significant. All the analyses and data visualization were performed in R V4.0.3 with following packages: phyloseq, vegan, tidyverse, dplyr, glm, ppcor, pairwise.adonis, pROC. NBZIMM, ggplot2, ggpubr, ComplexHeatmap, circlize and Hmisc.

### Data Availability

Raw sequence reads are deposited under BioProject PRJEB48269 and are associated with FIGS. 1b-d, 2a, 3b-c and FIGS. 6 a-b, 7 a-b, 8, 9, 10, 11, 12, and 13 a-h. Gut microbial motility was calculated based on species relative abundance and motility phenotype (GOLD database (v202109) and IJSEM database).

TABLE 7
Baseline characteristics of the study population

| | Overall | BNT162b2 | CoronaVac | |
|---|---|---|---|---|
| Variable | (N = 138) | (N = 101) | (N = 37) | p-value |
| Characteristics | | | | |
| Age, years, (Median (IQR)) | 47 (31.2, 55.0) | 42 (29.0, 53.0) | 55 (44.0, 57.0) | 0.003 |
| Female¹ | 93 (67.9) | 65 (65.0) | 28 (75.7) | 0.304 |
| BMI, kg/m², (Median (IQR)) | 21.8 (20.2, 24.5) | 21.8 (20.1, 24.6) | 22.2 (20.4, 23.7) | 0.946 |
| Overweight or Obese² | 53 (38.7) | 38 (38.0) | 15 (40.5) | 0.844 |
| Obese² | 27 (19.7) | 22 (22.0) | 5 (13.5) | 0.338 |
| Presence of Comorbidity | | | | |
| Hypertension | 14 (10.1) | 7 (6.9) | 7 (18.9) | 0.055 |
| Diabetes mellitus | 4 (2.9) | 3 (3.0) | 1 (2.7) | 1.000 |
| Allergy ever | 49 (35.5) | 40 (39.6) | 9 (24.3) | 0.111 |
| Diarrhea (past 3 month | 55 (40.4) | 42 (42.0) | 13 (36.1) | 0.560 |
| to current) | | | | |
| Other comorbidities³ | 15 (10.9) | 13 (12.9) | 2 (5.4) | 0.354 |
| Current medication | | | | |
| Antibiotic intake past | 6 (4.3) | 6 (5.9) | 0 (0.0) | 0.192 |
| 3 month and/or currently | | | | |
| Hormone therapy | 4 (2.9) | 4 (4.0) | 0 (0.0) | 0.574 |
| Immunomodulator | 3 (2.2) | 3 (3.0) | 0 (0.0) | 0.564 |
| Probiotics | 18 (13.1) | 12 (12.0) | 6 (16.2) | 0.572 |
| Vaccination in the past year | 53 (38.7) | 38 (38.0) | 15 (40.5) | 0.844 |
| Dietary habit | | | | |
| Vegetarian | 1 (1.0) | 1 (1.0) | 0 (0.0) | 1.000 |
| Diet change during vaccination | 0 (0.0) | 0 (0.0) | 0 (0.0) | — |
| Alcohol intake (within 2 weeks | 31 (22.5) | 25 (24.8) | 6 (16.2) | 0.361 |
| prior to first vaccine dose) | | | | |
| Exercise | | | | |
| Regular exercise | 86 (62.3) | 62 (61.4) | 24 (64.9) | 0.843 |
| (Strenuous/moderate) | | | | |
| SARS-CoV-2 antibody response | | | | |
| AUC of Anti-RBD IgG | 7889.5 (3110.8, 9588.5) | 8696.0 (7628.0, 11048.0) | 1725.0 (1418.0, 2459.0) | <0.001 |
| (Median (IQR)) ⁴ | | | | |
| sVNT (>60%) | 116 (84.1) | 100 (99.0) | 16 (43.2) | <0.001 |
| sVNT (inhibition %) | 93.9 (79.7, 95.9) | 95.2 (92.1, 96.4) | 57.6 (42.1, 69.3) | <0.001 |
| (Median (IQR)) | | | | |
| Any adverse events⁵ | | | | |
| After the first dose | 116 (84.7) | 93 (93.0) | 23 (62.2) | <0.001 |
| After the second dose | 120 (87.6) | 95 (95.0) | 25 (67.6) | <0.001 |

Categorical data are presented as number (percentage) and continuous variables as median (Interquartile range). Within group valid percentages are shown.
¹One participant requested concealment of gender.
²BMI between 23.0 and 25.0 kg/m² is classified as overweight and BMI above 25.0 kg/m² is classified as obese.
³Any other comorbidities: asthma, depression, eczema, high cholesterol, systemic lupus erythematosus, attention deficit hyperactivity disorder.
⁴ Plasma IgG antibody binding to SARS-Cov-2 RBD was reported as area under the curve.
⁵Any e events: injection site pain/burn, fatigue fever, injection site swelling/pruritus/erythema/induration, myalgia, drowsiness, headache, chills, dizziness, arthralgia, loss of appetite, abdominal pain, rhinorrhea, sore throat, diarrhea, pruritus, coughing, constipation, abdominal distension, nausea, flushing, hypersensitivity, muscle spasms, nasal congestion, edema, vomiting, tremor, eyelid edema, nosebleeds, hyposmia, ocular congestion, low back pain, increase of appetite, muscle pain, rib pain, eyes pain, palpitations.
Abbreviations. AUC, area under the curve; BMI, Body Mass Index; DM, Diabetes mellitus; IQR, interquartile range; RBD, receptor-binding domain; SARS-CoV-2, Severe Acute Respiratory Syndrome CoroVirus 2; sVNT, SARS-CoV-2 Surrogate Virus Neutralization Test.

All patents, patent applications, and other publications, including GenBank Accession Numbers and equivalents, cited in this application are incorporated by reference in the entirety for all purposes.

TABLE 1
Association between participant demographics and immune response

| | BNT162b2 (N = 101) | | | | CoronaVac (N = 37) | | | |
|---|---|---|---|---|---|---|---|---|
| Variables | AUC of Anti-RBD IgG | P value | sVNT (200) | P value | AUC of Anti-RBD IgG | P value | sVNT (10) | P value |
| Characteristic | | | | | | | | |
| Age | −0.016 | 0.872 | 0.064 | 0.525 | −0.241 | 0.151 | −0.294 | 0.077 |
| Gender | | 0.131 | | 0.871 | | 0.768 | | 0.821 |
| Female | 9109.0 (7775.0, 11627.0) | | 42.6 (30.0, 56.2) | | 1687.5 (1450.5, 2535.0) | | 55.7 (43.9, 66.9) | |
| Male | 8422.0 (6798.0, 10024.0) | | 42.9 (35.1, 49.3) | | 1878.0 (1046.0, 2181.0) | | 66.5 (38.6, 78.9) | |
| Body mass index | −0.136 | 0.176 | −0.015 | 0.880 | −0.305 | 0.066 | −0.385 | 0.018 |
| Overweight/Obese | | 0.074 | | 0.577 | | 0.080 | | 0.069 |
| No | 9233.0 (7846.0, 11627.0) | | 42.7 (31.1, 56.2) | | 1739.0 (1486.0, 2835.0) | | 65.3 (42.1, 78.9) | |
| Yes | 8330.0 (7203.0, 9266.0) | | 41.2 (33.1, 52.2) | | 1558.0 (1179.5, 1962.5) | | 49.9 (41.4, 59.3) | |
| Obese | | 0.033 | | 0.644 | | 0.128 | | 0.140 |
| No | 9089.0 (7775.0, 11445.0) | | 40.9 (33.1, 53.9) | | 1739.0 (1450.5, 2599.0) | | 59.0 (43.9, 74.0) | |
| Yes | 7832.5 (6894.0, 8659.0) | | 44.0 (29.9, 54.5) | | 1541.0 (979.5, 1727.0) | | 44.3 (38.6, 47.6) | |
| Comorbidities | | | | | | | | |
| Hypertension | | 0.663 | | 0.683 | | 0.608 | | 0.954 |
| No | 8701.5 (7675.0, 11048.0) | | 42.7 (33.3, 53.9) | | 1627.0 (1353.0, 2459.0) | | 57.9 (38.6, 67.7) | |
| Yes | 8538.0 (6843.5, 10921.5) | | 29.9 (23.9, 63.3) | | 1878.0 (1642.5, 2384) | | 47.6 (45, 78.6) | |
| Diabetes mellitus | | 0.652 | | 0.803 | | — | | — |
| No | 8677.5 ( 7639.8, 10958.7) | | 42.7 ( 31.6, 54.2 ) | | 1725.0 (1450.5, 2416.0) | | 57.9 (43.2, 69.5) | |
| Yes | 10530.0 ( 8866.5, 11263.5) | | 39.5 (36.4, 52.9) | | NA | | NA | |
| Allergy ever | | 0.262 | | 0.301 | | 0.638 | | 0.768 |
| No | 9062.0 (7754.0, 11627.0) | | 42.6 (33.2, 56.4) | | 1627.0 (1450.5, 2416.0) | | 55.7 (36.5, 69.5) | |
| Yes | 8657.5 (7245.8 , 9999.7) | | 41.9 (29.3, 51.2) | | 1753.0 (1353.0, 2611.0) | | 59.7 (44.3, 65.9) | |
| Diarrhoea (past 3 | | 0.992 | | 0.175 | | 0.558 | | 1.000 |
| month to current) | | | | | | | | |
| No | 8616.0 ( 7699.0, 10322.7 ) | | 40.9 ( 29.9, 51.7) | | 1725.0 (1450.5, 2523.0) | | 53.8 ( 38.1, 68.5 ) | |
| Yes | 8785.0 ( 7124.0, 11609.3 ) | | 43.2 (35.9, 56.1 ) | | 1604.0 (1486.0,1878.0) | | 57.6 ( 45.6, 66.5 ) | |

TABLE 1
Association between participant demographics and immune response

| | BNT162b2 (N = 101) | | | | CoronaVac (N = 37) | | | |
|---|---|---|---|---|---|---|---|---|
| Variables | AUC of Anti-RBD IgG | P value | sVNT (200) | P value | AUC of Anti-RBD IgG | P value | sVNT (10) | P value |
| Other comorbidities | | 0.258 | | 0.663 | | 0.676 | | 0.300 |
| No | 8777.0 (7651.5, 11251.5) | | 42.7 (31.6, 54.4) | | 1725.0 ( 1450.5, 2523.0 ) | | 58.3 ( 43.2, 69.5 ) | |
| Yes | 8084.0 (7405.0, 9069.0) | | 39.4 (33.3, 52.6) | | 1555.0 ( 1300.5, 1809.5 ) | | 35.8 ( 26.9, 44.9) | |
| Current | | | | | | | | |
| medication | | | | | | | | |
| Antibiotic intake | | | | | | | | |
| (within 3 month and | | 0.477 | | 0.994 | | — | | — |
| up to vaccination) | | | | | | | | |
| No | 8659.0 (7398.0, 10869.5) | | 42.9 (32.1, 54.1) | | 1687.5 (1385.5, 2416.0) | | 55.7 (40.4, 68.5) | |
| Yes | 9331.0 (8162.0, 11058.0) | | 39.5 (37.9, 56.2) | | NA | | NA | |
| Hormonal therapy | | 0.207 | | 0.503 | | — | | — |
| No | 8696.0 (7675.0, 11058.0) | | 42.6 (33.3, 54.4) | | 1725.0 (1418.0, 2459.0) | | 57.6 (42.1, 69.3) | |
| Yes | 7808.5 (3968.5, 8954.5) | | 32.8 (11.5, 54.2) | | NA | | NA | |
| Immunotherapy | | 0.226 | | 0.153 | | — | | — |
| No | 8701.5 (7628.0, 11058.0) | | 42.7 (33.3, 54.5) | | 1725.0 (1418, 2459) | | 57.6 (42.1, 69.3) | |
| Yes | 8084.0 (4563.5, 8403.5) | | 31.1 (15.8, 37.1) | | NA | | NA | |
| Probiotics | | 0.507 | | 0.221 | | 0.532 | | 0.587 |
| No | 8683.0 (7398.0, 10679.0) | | 41.1 (29.9, 54.2) | | 1753.0 (1450.5, 2523.0) | | 57.6 (41.4, 74.0) | |
| Yes | 8676.0 (8215.5, 12360.5) | | 51.2 (37.0, 53.4) | | 1574.0 (1353.0, 1725.0) | | 54.4 (42.1, 59.7) | |
| Vaccination in the | | 0.144 | | 0.222 | | 0.795 | | 0.795 |
| past year | | | | | | | | |
| No | 8894.5 (7675.0, 11627.0) | | 43.6 (33.3, 53.9) | | 1687.5 (1418.0, 2459.0) | | 55.7 (38.6, 69.3) | |
| Yes | 8297.0 (7230.0, 9511.0) | | 39.3 (29.9, 54.5) | | 1727.0 ( 1385.5, 2480.0 ) | | 64.7 (57.7, 72.9) | |
| Dietary habit | | | | | | | | |
| Vegetarian | | — | | — | | — | | — |
| No | 8659.0 (7516.5, 10869.5) | | 42.6 (33.2, 54.4) | | 1725.0 (1418.0, 2459.0) | | 57.6 (42.1, 69.3) | |
| Yes | NA | | NA | | NA | | NA | |
| Dietary change | | — | | — | | — | | — |
| during vaccination | | | | | | | | |
| No | 8696.0 (7651.5, 10869.5) | | 42.6 (33.2, 54.4) | | 1725.0 (1418.0, 2459.0) | | 57.6 (42.1, 69.3) | |

TABLE 1
Association between participant demographics and immune response

| | BNT162b2 (N = 101) | | | | CoronaVac (N = 37) | | | |
|---|---|---|---|---|---|---|---|---|
| Variables | AUC of Anti-RBD IgG | P value | sVNT (200) | P value | AUC of Anti-RBD IgG | P value | sVNT (10) | P value |
| Yes | NA | | NA | | NA | | NA | |
| Alcohol intake | | | | | | | | |
| (within 2 weeks of | | 0.213 | | 0.266 | | 0.066 | | 0.481 |
| vaccination) | | | | | | | | |
| No | 8556.5 (7344.5, 10679.0) | | 41.1 (32.1, 53.5) | | 1650.0 (1333.0, 2277.0) | | 51.8 (40.4, 68.7) | |
| Yes | 9357.0 (8067.0, 11899.0) | | 44.9 (35.7, 56.2) | | 2471.5 (1568.0, 4274.0) | | 61.8 (53.8, 69.3) | |
| Exercise | | | | | | | | |
| Regular exercise | | | | | | | | |
| (Strenuous/ | | 0.942 | | 0.197 | | 0.404 | | 0.422 |
| moderate) | | | | | | | | |
| No | 8723.0 (7344.5, 10626.5) | | 41.0 (29.3, 51.6) | | 1313.0 (979.5, 2879.0) | | 49.9 (24.9, 69.3) | |
| Yes | 8677.5 (7675.0, 11445.0) | | 43.2 (33.3, 54.5) | | 1740.0 (1542.5, 2277.0) | | 57.9 (46.6, 68.7) | |
| Any adverse events | | | | | | | | |
| after vaccination | | | | | | | | |
| After first dose | | 0.995 | | 0.112 | | 0.243 | | 0.298 |
| No | 9062.0 (7810.5, 10699.5) | | 39.0 (24.1, 41.7) | | 1895.5 (1541, 2587.0) | | 65.3 (50.6, 67.7) | |
| Yes | 8659.0 (7405.0, 11048.0) | | 43.1 (33.3, 54.5) | | 1604.0 (1150, 2175.0) | | 51.8 (36.9, 69.5) | |
| After second dose | | 0.121 | | 0.887 | | 0.737 | | 0.835 |
| No | 9701.0 (9062.0, 12659.0) | | 44.1 (39.3, 46.4) | | 1634.0 (1385.5, 2325.5) | | 52.2 (48.7, 72.7) | |
| Yes | 8656.0 (7398.0, 10869.5) | | 42.6 (33.2, 54.4) | | 1725.0 (1486, 2459) | | 58.3 (38.6, 69.3) | |

Data are correlation coefficient or median (IQR).
NA, Not applicable

TABLE 2
Factors influencing the baseline gut microbiome of the participants

| Variables | Df | SumsOfSqs | MeanSqs | F. Model | R² | P value |
|---|---|---|---|---|---|---|
| Vaccine groups | 1 | 0.17335307 | 0.17335307 | 0.862931074 | 0.006305075 | 0.694 |
| Gender | 1 | 0.208147283 | 0.208147283 | 1.037007829 | 0.007622983 | 0.361 |
| OWOB | 1 | 0.231733764 | 0.231733764 | 1.154453596 | 0.008479 | 0.219 |
| Obese | 1 | 0.23754336 | 0.23754336 | 1.183649662 | 0.00869157 | 0.201 |
| Active Hypertension currently | 1 | 0.204290565 | 0.204290565 | 1.018087121 | 0.007430312 | 0.4 |
| DM currently | 1 | 0.176532299 | 0.176532299 | 0.878859166 | 0.006420708 | 0.66 |
| Allergy ever | 1 | 0.22353908 | 0.22353908 | 1.114798878 | 0.008130405 | 0.292 |
| Diarrhea (in the past 3 month and at the moment) | 1 | 0.322621815 | 0.322621815 | 1.614701367 | 0.011906536 | 0.017 |
| Any other comorbidities | 1 | 0.281072633 | 0.281072633 | 1.404684645 | 0.010222975 | 0.036 |
| Antibiotic intake past 3 month and/or currently | 1 | 0.284561175 | 0.284561175 | 1.422301262 | 0.010349858 | 0.046 |
| Hormone intake currently | 1 | 0.220665006 | 0.220665006 | 1.100349782 | 0.008025871 | 0.269 |
| Immune drug intake currently | 1 | 0.175480944 | 0.175480944 | 0.873591419 | 0.006382469 | 0.668 |
| Probiotic intake currently | 1 | 0.225624018 | 0.225624018 | 1.125538182 | 0.008268384 | 0.252 |
| Vaccination in the past year | 1 | 0.113797724 | 0.113797724 | 0.564462045 | 0.004163791 | 0.998 |
| Dietary habit | 1 | 0.156880744 | 0.156880744 | 0.77939718 | 0.005740173 | 0.878 |
| Alcohol intake (within 2 weeks prior to first | 1 | 0.238828656 | 0.238828656 | 1.191716793 | 0.008686507 | 0.172 |
| vaccine dose) | | | | | | |
| Regular exercise (strenuous/moderate) | 1 | 0.318259492 | 0.318259492 | 1.592705609 | 0.011575509 | 0.017 |
| sVNT-200 Q1 (within BNT162b2 vaccinees) | 1 | 0.2329584 | 0.2329584 | 1.143064175 | 0.011414312 | 0.246 |
| sVNT-10 >60% (within CoronaVac vaccinees) | 1 | 0.254442111 | 0.254442111 | 1.337742476 | 0.036814133 | 0.091 |
| AE after the first dose | 1 | 0.421510761 | 0.421510761 | 2.121512258 | 0.015471768 | 0.002 |
| AE after the second dose | 1 | 0.201213828 | 0.201213828 | 1.004482396 | 0.007385657 | 0.46 |
| No. of AE after the first dose | 3 | 0.731689735 | 0.243896578 | 1.223522584 | 0.026857046 | 0.062 |
| No. of AE after the second dose | 3 | 0.564293266 | 0.188097755 | 0.937683781 | 0.020712673 | 0.667 |
| Age | 1 | 0.238760764 | 0.238760764 | 1.191375055 | 0.008684038 | 0.177 |
| BMI | 1 | 0.280693939 | 0.280693939 | 1.400894948 | 0.010270423 | 0.044 |
| Bacterial motility | 1 | 0.810878559 | 0.810878559 | 4.132897679 | 0.029492701 | 0.001 |
| Baseline observed species | 1 | 0.838276756 | 0.838276756 | 4.276932721 | 0.030489209 | 0.001 |
| Baseline Simpson diversity | 1 | 1.067447021 | 1.067447021 | 5.493400295 | 0.038824428 | 0.001 |
| Baseline Shannon diversity | 1 | 0.935264127 | 0.935264127 | 4.789192895 | 0.034016765 | 0.001 |

AE, adverse effect(s).
P values were given by PERMANOVA.

SUPPLEMENTARY TABLE 3
Difference of Dietary habit at baseline and one-
month after the second dose of vaccine (n = 72)

| Dietary intake (consumed | | | One-month after 2^{nd} | |
|---|---|---|---|---|
| in the past week) | | Baseline | dose of vaccine | p value |
| 1 | Pasta, pasta salad, | 70 | 70 | 1.00 |
| | or noodles | | | |
| 2 | Bread | 69 | 69 | 1.00 |
| 3 | Sweet baked foods | 66 | 66 | 1.00 |
| 4 | Meats | 71 | 71 | 1.00 |
| 5 | Seafood | 66 | 66 | 1.00 |
| 6 | Vegetables | 72 | 72 | N/A |
| 7 | Fruits and products | 72 | 72 | N/A |
| 8 | Milk and dairy products | 58 | 61 | 0.25 |
| 9 | Soup | 70 | 70 | 1.00 |
| 10 | Tea and coffee | 63 | 62 | 1.00 |
| 11 | Sugars and syrups | 56 | 56 | 1.00 |
| 12 | Alcoholic drinks | 15 | 18 | 0.25 |
| 13 | Vitamin pills | 22 | 21 | 1.00 |

SUPPLEMENTARY TABLE 4
Mixed effect models for persistently differentially abundant
species between low responders and responders

| | Baseline abundance | | One-month abundance | | Model 1 | | Model 2 | |
|---|---|---|---|---|---|---|---|---|
| | Low- | High- | Low- | High- | Effect | P | Effect | P |
| Biomarker | responders | responders | responders | responders | size | value | size | value |
| CoronaVac | | | | | | | | |
| Bifidobacterium | 0e+00 | 9.65e−03 | 0e+00 | 2.43e−03 | 0.109 | 0.011 | 0.109 | 0.023 |
| adolescentis | (0e+00, | (4.03e−02, | (0e+00, | (8.06e−03, | (0.027, | | (0.016, | |
| | 7.03e−03) | 8.46e−02) | 1.96e−03) | 3.7e−02) | 0.192) | | 0.203) | |
| Adlercreutzia | 6.29e−05 | 3.17e−03 | 0e+00 | 4.17e−04 | 0.02 | 0.096 | 0.019 | 0.148 |
| equolifaciens | (5.7e−04, | (3.64e−03, | (8.52e−05, | (6.08e−04, | (−0.004, | | (−0.007, | |
| | 3.15e−03) | 6.21e−03) | 7.12e−04) | 2.18e−03) | 0.044) | | 0.046) | |
| Asaccharobacter | 0e+00 | 8.7e−04 | 0e+00 | 7.01e−05 | 0.011 | 0.083 | 0.011 | 0.127 |
| celatus | (1.27e−04, | (1.25e−03, | (0e+00, | (2.57e−04, | (−0.002, | | (−0.003, | |
| | 8.02e−04) | 2.29e−03) | 2.59e−04) | 5.72e−04) | 0.024) | | 0.025) | |
| Ruminococcus | 0e+00 | 0e+00 | 0e+00 | 0e+00 | 0.011 | 0.048 | 0.007 | 0.257 |
| sp CAG 330 | (0e+00, | (0e+00, | (0e+00, | (0e+00, | (0, 0.022) | | (−0.005, | |
| | 0e+00) | 0e+00) | 0e+00) | 0e+00) | | | 0.019) | |
| Mitsuokella | 0e+00 | 0e+00 | 0e+00 | 0e+00 | 0.01 | 0.025 | 0.004 | 0.384 |
| multacida | (0e+00, | (0e+00, | (0e+00, | (0e+00, | (0.001, | | (−0.005, | |
| | 0e+00) | 0e+00) | 0e+00) | 0e+00) | 0.018) | | 0.012) | |
| Bacteroides | 1.5e−02 | 4.31e−03 | 3.81e−02 | 5.97e−03 | −0.089 | 0.034 | −0.104 | |
| vulgatus | (5.4e−02, | (9.7e−03, | (6.23e−02, | (2.82e−02, | (−0.17, | | (−0.196, | |
| | 7.37e−02) | 4.11e−02) | 1.59e−01) | 4.05e−02) | −0.007) | | −0.012) | |
| BNT162b2 | | | | | | | | |
| Bacteroides | 0e+00 | 0e+00 | 0e+00 | 0e+00 | 0.016 | 0.002 | 0.015 | |
| sp OM05 12 | (0e+00, | (0e+00, | (0e+00, | (0e+00, | (0.006, | | (0.005, | |
| | 0e+00) | 1.06e−04) | 0e+00) | 2.38e−04) | 0.026) | | 0.025) | |
| Bacteroides | 6.15e−04 | 1.96e−03 | 1.18e−03 | 3.6e−03 | 0.044 | 0.001 | 0.045 | 0.001 |
| thetaiotaomicron | (2.11e−03, | (6.55e−03, | (4.17e−03, | (6.2e−03, | (0.018, | | (0.02, | |
| | 6.63e−03) | 1.5e−02) | 7.59e−03) | 1.91e−02) | 0.07) | | 0.071) | |
| Fusobacterium | 0e+00 | 0e+00 | 0e+00 | 0e+00 | −0.011 | 0.061 | −0.011 | 0.061 |
| mortiferum | (0e+00, | (0e+00, | (0e+00, | (0e+00, | (−0.022, | | (−0.022, | |
| | 0e+00) | 0e+00) | 0e+00) | 0e+00) | 0.001) | | 0) | |

Model 1 is a crude model; Model 2 adjusted for age and time difference between sample collections.

TABLE 5
Adverse events after the first dose and second dose

| | After 1st dose | | | After 2nd dose | | |
|---|---|---|---|---|---|---|
| | BNT162b2 | Corona Vac | | BNT162b2 | Corona Vac | |
| Adverse events | (N = 101) | (N = 37) | p-value | (N = 101) | (N = 37) | p-value |
| Injection site pain/burn | 84 (84.0) | 17 (45.9) | <0.001 | 79 (79.0) | 16 (43.2) | <0.001 |
| Fatigue | 33 (33.0) | 3 (8.1) | 0.004 | 49 (49.0) | 6 (16.2) | <0.001 |
| Fever | 6 (6.0) | 1 (2.7) | 0.674 | 27 (27.0) | 0 (0.0) | <0.001 |
| Injection site swelling, pruritus, | 26 (26.0) | 1 (2.7) | 0.001 | 28 (28.0) | 6 (16.2) | 0.186 |
| erythema, induration | | | | | | |
| Myalgia | 22 (22.0) | 2 (5.4) | 0.023 | 26 (26.0) | 2 (5.4) | 0.008 |
| Drowsiness | 17 (17.0) | 3 (8.1) | 0.277 | 20 (20.0) | 2 (5.4) | 0.039 |
| Headache | 11 (11.0) | 1 (2.7) | 0.180 | 25 (25.0) | 0 (0.0) | <0.001 |
| Chills | 3 (3.0) | 0 (0.0) | 0.563 | 15 (15.0) | 1 (2.7) | 0.069 |
| Dizziness | 6 (6.0) | 1 (2.7) | 0.674 | 12 (12.0) | 1 (2.7) | 0.185 |
| Arthralgia | 4 (4.0) | 0 (0.0) | 0.574 | 10 (10.0) | 0 (0.0) | 0.062 |
| Loss of appetite | 2 (2.0) | 1 (2.7) | 1.000 | 7 (7.0) | 0 (0.0) | 0.189 |
| Abdominal pain | 1 (1.0) | 0 (0.0) | 1.000 | 6 (6.0) | 0 (0.0) | 0.190 |
| Rhinorrhea | 1 (1.0) | 1 (2.7) | 0.469 | 5 (5.0) | 2 (5.4) | 1.000 |
| Sore throat | 1 (1.0) | 0 (0.0) | 1.000 | 6 (6.0) | 0 (0.0) | 0.190 |
| Diarrhea | 2 (2.0) | 1 (2.7) | 1.000 | 5 (5.0) | 1 (2.7) | 1.000 |
| Pruritus | 3 (3.0) | 2 (5.4) | 0.612 | 4 (4.0) | 2 (5.4) | 0.661 |
| Coughing | 1 (1.0) | 0 (0.0) | 1.000 | 4 (4.0) | 0 (0.0) | 0.574 |
| Constipation | 0 (0.0) | 0 (0.0) | — | 3 (3.0) | 0 (0.0) | 0.563 |
| Abdominal distension | 0 (0.0) | 0 (0.0) | — | 3 (3.0) | 0 (0.0) | 0.563 |
| Nausea | 1 (1.0) | 0 (0.0) | 1.000 | 3 (3.0) | 0 (0.0) | 0.563 |
| Flushing | 1 (1.0) | 0 (0.0) | 1.000 | 2 (2.0) | 0 (0.0) | 1.000 |
| Hypersensitivity | 1 (1.0) | 0 (0.0) | 1.000 | 1 (1.0) | 1 (2.7) | 0.469 |
| Muscle spasms | 1 (1.0) | 0 (0.0) | 1.000 | 1 (1.0) | 1 (2.7) | 0.469 |
| Nasal Congestion | 0 (0.0) | 0 (0.0) | — | 2 (2.0) | 0 (0.0) | 1.000 |
| Edema | 0 (0.0) | 0 (0.0) | — | 1 (1.0) | 0 (0.0) | 1.000 |
| Vomiting | 0 (0.0) | 0 (0.0) | — | 0 (0.0) | 0 (0.0) | — |
| Tremor | 0 (0.0) | 0 (0.0) | — | 0 (0.0) | 0 (0.0) | — |
| Eyelid edema | 0 (0.0) | 0 (0.0) | — | 0 (0.0) | 0 (0.0) | — |
| Nosebleeds | 0 (0.0) | 0 (0.0) | — | 1 (1.0) | 0 (0.0) | 1.000 |
| Hyposmia | 0 (0.0) | 0 (0.0) | — | 0 (0.0) | 0 (0.0) | — |
| Ocular congestion | 1 (1.0) | 0 (0.0) | 1.000 | 0 (0.0) | 0 (0.0) | |
| Others¹ | 12 (12.0) | 1 (2.70) | 0.185 | 15 (15.0) | 1 (2.70) | 0.069 |
| Number of adverse events (listed) | | | <0.001 | | | <0.001 |
| 0 | 7 (7.0) | 14 (37.8) | | 5 (5.0) | 12 (32.4) | |
| 1 | 39 (39.0) | 16 (43.2) | | 22 (22.0) | 15 (40.5) | |
| 2 | 21 (21.0) | 4 (10.8) | | 19 (19.0) | 5 (13.5) | |
| >=3 | 33 (33.0) | 3 (8.11) | | 54 (54.0) | 5 (13.5) | |
| Any adverse events | 93 (93.0) | 23 (62.2) | <0.001 | 95 (95.0) | 25 (67.6) | <0.001 |

Data are n (%).
Within group valid percentages are shown. There is one missing data in BNT162b2 group.
¹Others include low back pain, increase of appetite, muscle pain, rib pain, eyes pain, palpitations.

TABLE 6
Logistic regression models for having adverse effect after the first vaccine dose based
on cluster membership

| | | Model 1 | | | | Model 2 | | | |
|---|---|---|---|---|---|---|---|---|---|
| Vaccine group | Dose | OR | 2.50% | 97.50% | p | OR | 2.50% | 97.50% | p |
| BNT162b2 | 1 | 6.93 | 1.41 | 34.19 | 0.017 | 13.78 | 2.48 | 76.6 | 0.003 |
| (Cluster 2 vs. Cluster 1) | 2 | 7.41 | 1.14 | 47.96 | 0.036 | 9.55 | 1.35 | 67.43 | 0.024 |
| CoronaVac | 1 | 6.67 | 1.34 | 33.12 | 0.02 | | | | |
| (Cluster 1 vs. Cluster 2) | 2 | 1.58 | 0.35 | 7.17 | 0.551 | | | | |

Model 1 is a crude model;
Model 2 adjusted for alcohol drinking within 2 weeks prior to the 1st vaccine dose, which was significantly associated with cluster membership in BNT162b2 vaccinees.

TABLE 7
The association between immunity response and Adverse events after first dose (Overall)

| | BNT162b2 (N = 101) | | | | Corona Vac (N = 37) | | | |
|---|---|---|---|---|---|---|---|---|
| Adverse events | AUC of Anti-RBD IgG | P value | sVNT (200) | P value | AUC of Anti-RBD IgG | P value | sVNT (10) | P value |
| Any adverse events after | | 0.995 | | 0.112 | | 0.244 | | |
| first dose | | | | | | | | 0.298 |
| No | 9062.0 (7810.5, 10699.5) | | 39.0 (24.1, 41.7) | | 1895.5 (1541.0, 2587.0) | | 65.3 (50.6, 67.7) | |
| Yes | 8659.0 (7405.0, 11048.0) | | 43.1 (33.3, 54.5) | | 1604.0 (1150.0, 2175.0) | | 51.8 (36.9, 69.5) | |
| Injection site pain/burn | | 0.898 | | 0.206 | | 0.424 | | 0.478 |
| No | 8497.5 (7459.0, 11880.0) | | 39.2 (34.5, 45.3) | | 1726.0 (1542.5, 2523.0) | | 62.2 (46.6, 67.1) | |
| Yes | 8677.5 (7516.5, 10679.0) | | 43.2 (32.2, 55.4) | | 1604.0 (1046.0, 2373.0) | | 51.8 (24.9, 69.6) | |
| Fatigue | | 0.593 | | 0.032 | | 0.244 | | 0.052 |
| No | 8656.0 (7398.0, 10869.5) | | 39.4 (29.9, 52.6) | | 1740.0 (1418.0, 2587.0) | | 59.0 (44.3, 69.6) | |
| Yes | 8723.0 (7732.0, 11058.0) | | 44.9 (40.1, 56.5) | | 1544.0 (1295.0, 1551.0) | | 35.2 (26.5, 40.4) | |
| Fever | | 0.499 | | 0.046 | | — | | — |
| No | 8657.5 (7405.0, 11058.0) | | 41.2 (33.1, 52.6) | | 1726.0 (1450.5, 2523.0) | | 57.9 (43.2, 69.5) | |
| Yes | 10408.5 (8347.0, 10667.0) | | 58.1 (51.4, 60.9) | | NA | | NA | |
| Injection site swelling, | | 0.398 | | 0.162 | | 0.758 | | 0.486 |
| pruritus, erythema, | | | | | | | | |
| induration | | | | | | | | |
| No | 9065.5 (7628.0, 11627.0) | | 40.9 (31.1, 52.6) | | 1687.5 (1385.5, 2523.0) | | 55.7 (40.4, 68.5) | |
| Yes | 8301.5 (7229.0, 10287.0) | | 48.4 (36.1, 56.5) | | | | | |
| Myalgia | | 0.163 | | 0.825 | | 0.036 | | 0.090 |
| No | 9065.5 (7732.0, 11627.0) | | 42.0 (33.3, 54.4) | | 1727.0 (1484.5, 2523) | | 58.3 (45, 69.5) | |
| Yes | 8384.5 (7229.0, 9266.0) | | 47.4 (28.7, 54.5) | | 1036.9 (819.7, 1254) | | 29.6 (17.1, 42.1) | |
| Drowsiness | | 0.233 | | 0.121 | | 0.292 | | 0.267 |
| No | 8538.0 (7398.0, 10869.5) | | 41.2 (32.1, 52.6) | | 1627.0 (1353, 2459) | | 52.8 (38.6, 69.3) | |
| Yes | 9402.0 (8347.0, 12084) | | 52.0 (40.8, 55.5) | | 1753.0 (1739, 3614.5) | | 64.7 (62.2, 74.6) | |
| Headache | | 0.092 | | 0.783 | | — | | — |
| No | 8847.0 (7732.0, 11058.0) | | 42.9 (33.1, 54.4) | | 1726.0 (1385.5, 2523.0) | | 57.9 (40.4, 69.5) | |
| Yes | 7675.0 (6676.0, 8535.0) | | 41.3 (38.3, 50.2) | | NA | | NA | |
| Chills | | 0.793 | | 0.163 | | — | | — |
| No | 8696.0 (7628.0, 11048.0) | | 42.6 (33.1, 53.9) | | 1725.0 (1418.0, 2459.0) | | 57.6 (42.1, 69.3) | |

TABLE 7
The association between immunity response and Adverse events after first dose (Overall)

| | BNT162b2 (N = 101) | | | | CoronaVac (N = 37) | | | |
|---|---|---|---|---|---|---|---|---|
| Adverse events | AUC of Anti-RBD IgG | P value | sVNT (200) | P value | AUC of Anti-RBD IgG | P value | sVNT (10) | P value |
| Yes | 8347 .0 (7620.5, 9702.5) | | 57.8 (48.9, 61.5) | | NA | | NA | |
| Dizziness | | 0.429 | | 0.372 | | — | | — |
| No | 8701.5 (7628.0, 11445.0) | | 42.0 (33.3, 52.6) | | 1687.5 (1385.5, 2416.0) | | 55.7 (40.4, 68.5) | |
| Yes | 8301.5 (7229.0, 9476.0) | | 56.5 (28.4, 57.8) | | NA | | NA | |
| Arthralgia | | 0.100 | | 0.937 | | — | | — |
| No | 8709.5 (7658.0, 11251.5) | | 42.7 (33.2, 54.4) | | 1725.0 (1418.0, 2459.0) | | 57.6 (42.1, 69.3) | |
| Yes | 7452.0 (6379.5, 8191.0) | | 41.6 (33.9, 50.2) | | NA | | NA | |
| Loss of appetite | | 0.951 | | 0.284 | | — | | — |
| No | 8677.5 (7405.0, 11058.0) | | 42.0 (33.1, 54.4) | | 1726.0 (1450.5, 2523.0) | | 57.9 (43.2, 69.5) | |
| Yes | 8597.0 (8347.0, 8847.0) | | 51.3 (44.9, 57.8) | | NA | | NA | |
| Abdominal pain | | 0.253 | | 0.556 | | — | | — |
| No | 8659.0 (7516.5, 10869.5) | | 42.6 (33.2, 54.4) | | 1725.0 (1418.0, 2459.0) | | 57.6 (42.1, 69.3) | |
| Yes | | | | | NA | | NA | |
| Rhinorrhea | | 0.146 | | 0.268 | | — | | — |
| No | 8696.0 (7651.5, 11053.0) | | 42.9 (33.3, 54.4) | | 1726.0 (1450.5, 2523.0) | | 57.9 (43.2, 69.5) | |
| Yes | | | | | NA | | NA | |
| Sore throat | | 0.234 | | 0.917 | | — | | — |
| No | 8659.0 (7516.5, 10869.5) | | 42.9 (33.2, 54.4) | | 1725.0 (1418.0, 2459.0) | | 57.6 (42.1, 69.3) | |
| Yes | | | | | NA | | NA | |
| Diarrhea | | 0.530 | | 0.331 | | — | | — |
| No | 8677.5 (7628.0, 11058.0) | | 42.0 (33.1, 54.4) | | 1726.0 (1450.5, 2523.0) | | 57.9 (43.2, 69.5) | |
| Yes | 8038.0 (7229.0, 8847.0) | | 50.7 (44.9, 56.5) | | NA | | NA | |
| Pruritus | | 0.856 | | 0.746 | | 0.330 | | 0.126 |
| No | 8659.0 (7628.0, 11048.0) | | 42.6 (33.1, 54.4) | | 1650.0 (1385.5, 2416.0) | | 53.8 (40.4, 67.1) | |
| Yes | 8723.0 (7504.0, 10175.0) | | 43.2 (39.6, 50.0) | | 2294.0 (1977.0, 2611.0) | | 75.3 (69.6, 81.0) | |
| Coughing | | — | | — | | — | | — |
| No | 8696.0 (7651.5, 11053.0) | | 42.6 (33.2, 54.4) | | 1725.0 (1418.0, 2459.0) | | 57.6 (42.1, 69.3) | |
| Yes | NA | | NA | | NA | | NA | |

TABLE 7
The association between immunity response and Adverse events after first dose (Overall)

| | BNT162b2 (N = 101) | | | | CoronaVac (N = 37) | | | |
|---|---|---|---|---|---|---|---|---|
| Adverse events | AUC of Anti-RBD IgG | P value | sVNT (200) | P value | AUC of Anti-RBD IgG | P value | sVNT (10) | P value |
| Constipation | | — | | — | | — | | — |
| No | 8677.5 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Abdominal distension | | — | | — | | — | | — |
| No | 8677.5 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Nausea | | — | | — | | — | | — |
| No | 8696.0 (7651.5, 11053.0) | | 42.6 (33.2, 54.1) | | 1725.0 (1418.0, 2459.0) | | 57.6 (42.1, 69.3) | |
| Yes | NA | | NA | | NA | | NA | |
| Flushing | | — | | — | | — | | — |
| No | 8696.0 (7516.5, 11053.0) | | 42.6 (33.2, 54.4) | | 1725.0 (1418.0, 2459.0) | | 57.6 (42.1, 69.3) | |
| Yes | NA | | NA | | NA | | NA | |
| Hypersensitivity | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Muscle spasms | | — | | — | | — | | — |
| No | 8696.0 (7516.5, 11053.0) | | 42.9 (33.2, 54.4) | | 1725.0 (1418.0, 2459.0) | | 57.6 (42.1, 69.3) | |
| Yes | NA | | NA | | NA | | NA | |
| Nasal Congestion | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Edema | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Vomiting | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Tremor | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |

TABLE 7
The association between immunity response and Adverse events after first dose (Overall)

| | BNT162b2 (N = 101) | | | | CoronaVac (N = 37) | | | |
|---|---|---|---|---|---|---|---|---|
| Adverse events | AUC of Anti-RBD IgG | P value | sVNT (200) | P value | AUC of Anti-RBD IgG | P value | sVNT (10) | P value |
| Yes | NA | | NA | | NA | | NA | |
| Eyelid edema | | | | — | | | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Nosebleeds | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Hyposmia | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Ocular congestion | | — | | — | | — | | — |
| No | 8696.0 (7516.5, 11053.0) | | 42.9 (33.3, 54.4) | | 1725.0 (1418.0, 2459.0) | | 57.6 (42.1, 69.3) | |
| Yes | NA | | NA | | NA | | NA | |
| Number of adverse | | 0.933 | | 0.018 | | 0.139 | | 0.204 |
| events (listed) | | | | | | | | |
| 0 | 9062.0 (7810.5, 10699.5) | | 39.0 (24.1, 41.7) | | 1895.5 (1541.0, 2587.0) | | 65.3 (50.6, 67.7) | |
| 1 | 8659.0 (7398.0, 11356.0) | | 39.4 (30.5, 53.5) | | 1739.0 (1399.5, 2492.0) | | 57.9 (39.7, 73.8) | |
| 2 | 8238.0 (7298.0, 9476.0) | | 43.1 (29.9, 50.0) | | 1150.0 (1012.8, 1406.0) | | 40.4 (28.2, 43.9) | |
| >=3 | 8707.0 (7994.0, 11058.0) | | 51.0 (40.1, 56.5) | | 1977.0 (1398.4, 3726.5) | | 69.6 (43.4, 77.1) | |
| Others¹ | | 0.429 | | 0.337 | | — | | — |
| No | 8715.0 (7651.5, 11251.5) | | 42.0 (32.1, 54.1) | | 1687.5 (1385.5, 2523.0) | | 57.9 (40.4, 69.5) | |
| Yes | 8301.5 (6861.0, 9497.5) | | 47.4 (37.8, 57.1) | | NA | | NA | |

Data are median (IQR).
NA, Not applicable. There is one missing data in BNT162b2 group.
¹Others include low back pain, increase of appetite, muscle pain, rib pain, eyes pain, palpitations.

TABLE 8
The association between immunity response and Adverse events after second dose (Overall)

| | BNT162b2 (N = 101) | | | | CoronaVac (N = 37) | | | |
|---|---|---|---|---|---|---|---|---|
| Adverse events | AUC of Anti-RBD IgG | p-value | sVNT (200) | p-value | AUC of Anti-RBD IgG | p-value | sVNT (10) | p-value |
| Any adverse events | | 0.121 | | 0.887 | | 0.737 | | 0.835 |
| after second dose | | | | | | | | |
| No | 9701.0 (9062.0, 12659.0) | | 44.1 (39.3, 46.4) | | 1634.0 (1385.5, 2325.5) | | 52.2 (48.7, 72.7) | |
| Yes | 8656.0 (7398.0, 10869.5) | | 42.6 (33.2, 54.4) | | 1725.0 (1486.0, 2459.0) | | 58.3 (38.6, 69.3) | |
| Injection site | | 0.986 | | 0.213 | | 0.867 | | 0.797 |
| pain/burn | | | | | | | | |
| No | 8576.0 (7732.0, 11664.0) | | 37.0 (33.1, 51.4) | | 1725.0 (1483.0, 2181.0) | | 53.8 (45.7, 66.5) | |
| Yes | 8696.0 (7516.5, 10679.0) | | 43.2 (33.3, 55.4) | | 1776.0 (1150.0, 2857.0) | | 57.9 (31.3, 69.5) | |
| Fatigue | | 0.352 | | 0.098 | | 0.363 | | 0.533 |
| No | 8659.0 (7398.0, 9973.0) | | 40.1 (29.9, 52.6) | | 1753.0 (1385.5, 2599.0) | | 58.3 (43.2, 69.5) | |
| Yes | 8707.0 (7732.0, 11698.0) | | 43.9 (37, 55.5) | | 1551.0 (1486.0, 1650.0) | | 51.6 (35.2, 66.1) | |
| Fever | | 0.347 | | 0.119 | | — | | — |
| No | 8538.0 (7391.0, 10691.0) | | 41.2 (29.9, 52.6) | | 1725.0 (1418.0, 2459.0) | | 57.6 (42.1, 69.3) | |
| Yes | 9402.0 (7998.0, 11251.5) | | 48.3 (36.1, 56.7) | | NA | | NA | |
| Injection site | | | | | | | | |
| swelling, pruritus, | | 0.844 | | 0.078 | | 0.342 | | 0.857 |
| erythema, | | | | | | | | |
| induration | | | | | | | | |
| No | 8715.0 (7516.5, 10869.5) | | 41.1 (30.5, 52.4) | | 1604.0 (1385.5, 2416.0) | | 53.8 (40.4, 73.8) | |
| Yes | 8479.5 (7284.5, 11251.5) | | 48.4 (36.2, 57.4) | | 1927.5 (1650.0, 2835.0) | | 66.0 (44.3, 67.7) | |
| Myalgia | | 0.623 | | 0.517 | | 0.817 | | 0.769 |
| No | 8785.0 (7628.0, 11627.0) | | 43.1 (33.3, 55.2) | | 1727.0 (1385.5, 2523.0) | | 57.6 (40.4, 69.5) | |
| Yes | 8556.5 (7405.0, 9904.0) | | 40.3 (28.7, 52.5) | | 1641.5 (1558.0, 1725.0) | | 52.7 (45.7, 59.7) | |
| Drowsiness | | 0.676 | | 0.250 | | 0.108 | | 0.048 |
| No | 8616.0 (7540.0, 10869.5) | | 41.2 (30.5, 53.5) | | 1650.0 (1385.5, 2416.0) | | 53.8 (40.4, 67.1) | |
| Yes | 8777.0 (7261.0, 11764.5) | | 46.6 (40.0, 55.4) | | 3828.5 (2181.0, 5476.0) | | 81.9 (79.3, 84.6) | |
| Headache | | 0.702 | | 0.937 | | — | | — |
| No | 8707.0 (7710.0, 11159.0) | | 43.1 (33.2, 53.5) | | 1725.0 (1418.0, 2459.0) | | 57.6 (42.1, 69.3) | |

TABLE 8
The association between immunity response and Adverse events after second dose (Overall)

| | BNT162b2 (N = 101) | | | | CoronaVac (N = 37) | | | |
|---|---|---|---|---|---|---|---|---|
| Adverse events | AUC of Anti-RBD IgG | p-value | sVNT (200) | p-value | AUC of Anti-RBD IgG | p-value | sVNT (10) | p-value |
| Yes | 8422.0 (7298.0, 11048.0) | | 41.3 (36.3, 54.4) | | NA | | NA | |
| Chills | | 0.025 | | 0.311 | | — | | — |
| No | 8537.0 (7298.0, 10586.0) | | 41.3 (33.1, 52.6) | | 1687.5 (1385.5, 2523.0) | | 55.7 (40.4, 68.5) | |
| Yes | 10245.0 (8884.0, 11812.0) | | 50.0 (36.7, 56.2) | | NA | | NA | |
| Dizziness | | 0.820 | | 0.311 | | — | | — |
| No | 8657.5 (7540.0, 10874.5) | | 42.0 (32.1, 52.6) | | 1687.5 (1385.5, 2416.0) | | 55.7 (40.4, 68.5) | |
| Yes | 9091.5 (7428.5, 11337.5) | | 49.0 (36.3, 57.3) | | NA | | NA | |
| Arthralgia | | 0.850 | | 0.690 | | — | | — |
| No | 8677.5 (7628.0, 11058.0) | | 42.7 (33.3, 54.5) | | 1725.0 (1418.0, 2459.0) | | 57.6 (42.1, 69.3) | |
| Yes | 8681.5 (7405.0, 9904.0) | | 42.2 (28.4, 52.5) | | NA | | NA | |
| Loss of appetite | | 0.317 | | 0.317 | | — | | — |
| No | 8659.0 (7391.0, 11048.0) | | 41.3 (33.1, 54.4) | | 1725.0 (1418.0, 2459.0) | | 57.6 (42.1, 69.3) | |
| Yes | 8847.0 (8501.5, 10674.5) | | 52.2 (42.7, 53.9) | | NA | | NA | |
| Abdominal pain | | 0.744 | | 0.994 | | — | | — |
| No | 8677.5 (7628.0, 11048.0) | | 42.7 (33.1, 54.5) | | 1725.0 (1418.0, 2459.0) | | 57.6 (42.1, 69.3) | |
| Yes | 8949.0 (6519.0, 11445.0) | | 44.5 (35.7, 52.2) | | NA | | NA | |
| Rhinorrhea | | 0.601 | | 0.236 | | 0.243 | | 0.300 |
| No | 8659.0 (7516.5, 11053.0) | | 41.3 (33.2, 53.5) | | 1727.0 (1450.5, 2523.0) | | 58.3 (43.2, 69.5) | |
| Yes | 8256.0 (6519.0, 10287.0) | | 53.9 (48.3, 56.5) | | 1268.8 (979.5, 1558.0) | | 42.1 (38.6, 45.7) | |
| Sore throat | | 0.051 | | 0.856 | | — | | — |
| No | 8616.0 (7405.0, 10586.0) | | 43.0 (33.1, 54.5) | | 1725.0 (1418.0, 2459.0) | | 57.6 (42.1, 69.3) | |
| Yes | 12040.5 (11445.0, 12087.0) | | 40.8 (33.3, 53.9) | | NA | | NA | |
| Diarrhea | | 0.507 | | 0.420 | | — | | — |
| No | 8696.0 (7651.5, 10869.5) | | 43.1 (32.1, 54.5) | | 1726.0 (1450.5, 2523.0) | | 57.9 (43.2, 69.5) | |
| Yes | 7846.0 (6519.0, 11445.0) | | 39.4 (33.3, 40.6) | | NA | | NA | |
| Pruritus | | 0.840 | | 0.718 | | 0.216 | | 0.270 |

TABLE 8
The association between immunity response and Adverse events after second dose (Overall)

| | BNT162b2 (N = 101) | | | | CoronaVac (N = 37) | | | |
|---|---|---|---|---|---|---|---|---|
| Adverse events | AUC of Anti-RBD IgG | p-value | sVNT (200) | p-value | AUC of Anti-RBD IgG | p-value | sVNT (10) | p-value |
| No | 8657.5 (7516.5, 11053.0) | | 42.0 (33.2, 54.4) | | 1650.0 (1385.5, 2277.0) | | 53.8 (40.4, 68.5) | |
| Yes | 8709.5 (7113.0, 10175.0) | | 47.6 (35.8, 54.4) | | 2535.0 (2459.0, 2611.0) | | 72.9 (64.7, 81.0) | |
| Coughing | | 0.329 | | 0.196 | | — | | — |
| No | 8657.5 (7516.5, 10869.5) | | 42.0 (32.1, 54.1) | | 1725.0 (1418.0, 2459.0) | | 57.6 (42.1, 69.3) | |
| Yes | 10672.5 (8403.0, 12651.5) | | 51.9 (44.2, 58.2) | | NA | | NA | |
| Constipation | | 0.031 | | 0.182 | | — | | — |
| No | 8707.0 (7688.0, 11058.0) | | 42.9 (33.3, 54.5) | | 1725.0 (1418.0, 2459.0) | | 57.6 (42.1, 69.3) | |
| Yes | 6519.0 (6024.5, 6962.0) | | 28.4 (17.0, 38.4) | | NA | | NA | |
| Abdominal | | 0.396 | | 0.443 | | — | | — |
| distension | | | | | | | | |
| No | 8696.0 (7628.0, 11058.0) | | 42.6 (33.1, 54.4) | | 1725.0 (1418.0, 2459.0) | | 57.6 (42.1, 69.3) | |
| Yes | 8347.0 (7433.0, 8527.0) | | 48.3 (43.9, 53.1) | | NA | | NA | |
| Nausea | | 0.031 | | 0.711 | | — | | — |
| No | 8656.0 (7405.0, 10667.0) | | 42.9 (33.1, 54.4) | | 1725.0 (1418.0, 2459.0) | | 57.6 (42.1, 69.3) | |
| Yes | 12084.0 (11764.5, 12873.5) | | 41.0 (40.8, 48.1) | | NA | | NA | |
| Flushing | | 0.740 | | 0.452 | | — | | — |
| No | 8677.5 (7405.0, 11058.0) | | 42.7 (33.3, 54.5) | | 1725.0 (1418.0, 2459.0) | | 57.6 (42.1, 69.3) | |
| Yes | 8358.5 (7994.0, 8723.0) | | 34.8 (26.4, 43.2) | | NA | | NA | |
| Hypersensitivity | | — | | — | | — | | — |
| No | 8696.0 (7516.5, 11053.0) | | 42.9 (33.3, 54.4) | | 1687.5 (1385.5, 2523.0) | | 55.7 (40.4, 68.5) | |
| Yes | NA | | NA | | NA | | NA | |
| Muscle spasms | | — | | — | | — | | — |
| No | 8659.0 (7516.5, 11053.0) | | 42.9 (33.2, 54.4) | | 1687.5 (1385.5, 2523.0) | | 55.7 (40.4, 68.5) | |
| Yes | NA | | NA | | NA | | NA | |
| Nasal Congestion | | 0.667 | | 0.853 | | — | | — |
| No | 8677.5 (7628.0, 11048.0) | | 42.7 (33.1, 54.4) | | 1725.0 (1418.0, 2459.0) | | 57.6 (42.1, 69.3) | |
| Yes | 8257.5 (4888.0, 11627.0) | | 45.0 (33.3, 56.8) | | NA | | NA | |

TABLE 8
The association between immunity response and Adverse events after second dose (Overall)

| | BNT162b2 (N = 101) | | | | CoronaVac (N = 37) | | | |
|---|---|---|---|---|---|---|---|---|
| Adverse events | AUC of Anti-RBD IgG | p-value | sVNT (200) | p-value | AUC of Anti-RBD IgG | p-value | sVNT (10) | p-value |
| Edema | | — | | — | | — | | — |
| No | 8659.0 (7516.5, 11053.0) | | 42.9 (33.2, 54.4) | | 1725.0 (1418.0, 2459.0) | | 57.6 (42.1, 69.3) | |
| Yes | NA | | NA | | NA | | NA | |
| Vomiting | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Tremor | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Eyelid edema | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Nosebleeds | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Hyposmia | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Ocular congestion | | — | | — | | — | | — |
| No | 8659.0 (7516.5, 11053.0) | | 42.7 (33.2, 54.4) | | 1725.0 (1418.0, 2459.0) | | 57.6 (42.1, 69.3) | |
| Yes | NA | | NA | | NA | | NA | |
| Number of adverse | | 0.320 | | 0.064 | | 0.531 | | 0.702 |
| events (listed) | | | | | | | | |
| 0 | 9701.0 (9062.0, 12659.0) | | 44.1 (39.3, 46.4) | | 1634.0 (1385.5, 2325.5) | | 52.2 (48.7, 72.7) | |
| 1 | 8007.0 (6875.0, 9575.0) | | 41.9 (29.9, 52.6) | | 1725.0 (1399.0, 2416.0) | | 51.8 (36.5, 67.0) | |
| 2 | 8576.0 (6589.5, 10089.5) | | 35.9 (26.2, 45.8) | | 1650.0 (1486.0, 2835.0) | | 65.9 (57.6, 66.1) | |
| >=3 | 8715.0 (7846.0, 11445.0) | | 44.3 (39.4, 56.5) | | 1977.0 (1558.0, 2181.0) | | 69.6 (45.7, 79.3) | |

TABLE 8
The association between immunity response and Adverse events after second dose (Overall)

| | BNT162b2 (N = 101) | | | | CoronaVac (N = 37) | | | |
|---|---|---|---|---|---|---|---|---|
| Adverse events | AUC of Anti-RBD IgG | p-value | sVNT (200) | p-value | AUC of Anti-RBD IgG | p-value | sVNT (10) | p-value |
| Others¹ | | 0.595 | | 0.300 | | — | | — |
| No | 8707.0 (7675.0, 11058.0) | | 41.3 (31.1, 54.4) | | 1687.5 (1385.5, 2523.0) | | 57.9 (40.4, 69.5) | |
| Yes | 8347.0 (7415.5, 9748.5) | | 46.4 (37.8, 55.0) | | NA | | NA | |

Data are median (IQR).
NA, Not applicable.
There is one missing data in BNT162b2 group.
¹Others include low back pain, increase of appetite, muscle pain, rib pain, eyes pain, palpitations.

### References

1. Naaber P, Tserel L, Kangro K, et al. Dynamics of antibody response to BNT162b2 vaccine after six months: a longitudinal prospective study. Lancet Reg Health Eur 2021:100208. 2021/09/14. DOI: 10.1016/j.lanepe.2021.100208.
2. Thomas SJ, Moreira ED, Jr., Kitchin N, et al. Safety and Efficacy of the BNT162b2 mRNA Covid-19 Vaccine through 6 Months. N Engl J Med 2021 2021/09/16. DOI: 10.1056/NEJMoa2110345.
3 Khoury DS, Cromer D, Reynaldi A, et al. Neutralizing antibody levels are highly predictive of immune protection from symptomatic SARS-COV-2 infection. Nat Med 2021; 27:1205-1211. 2021/05/19. DOI: 10.1038/s41591-021-01377-8.
4. Lynn DJ, Benson SC, Lynn MA, et al. Modulation of immune responses to vaccination by the microbiota: implications and potential mechanisms. Nat Rev Immunol 2021 2021/05/19. DOI: 10.1038/s41577-021-00554-7.
5. Hagan T, Cortese M, Rouphael N, et al. Antibiotics-Driven Gut Microbiome Perturbation Alters Immunity to Vaccines in Humans. Cell 2019; 178:1313-1328 e1313. 2019/09/7. DOI: 10.1016/j.cell.2019.08.010.
6. New JS, Dizon BLP, Fucile CF, et al. Neonatal Exposure to Commensal-Bacteria-Derived Antigens Directs Polysaccharide-Specific B-1 B Cell Repertoire Development. Immunity 2020; 53:172-186 e176. 2020/07/2. DOI: 10.1016/j.immuni.2020.06.006.
7. Li H, Limenitakis JP, Greiff V, et al. Mucosal or systemic microbiota exposures shape the B cell repertoire. Nature 2020; 584:274-278. 2020/08/08. DOI: 10.1038/s41586-020-2564-6.
8. Tan CW, Chia WN, Qin X, et al. A SARS-COV-2 surrogate virus neutralization test based on antibody-mediated blockage of ACE2-spike protein-protein interaction. Nat Biotechnol 2020; 38:1073-1078. 2020/07/25. DOI: 10.1038/s41587-020-0631-z.
9. Droge W, Mannel D, Falk W, et al. Suppression of cytotoxic T lymphocyte activation by L-ornithine. J Immunol 1985; 134:3379-3383. 1985/05/01.
10. Turner JS, O′Halloran JA, Kalaidina E, et al. SARS-COV-2 mRNA vaccines induce persistent human germinal centre responses. Nature 2021; 596:109-113. 2021/06/29. DOI: 10.1038/s41586-021-03738-2.
11. Galvan-Pena S, Leon J, Chowdhary K, et al. Profound Treg perturbations correlate with COVID-19 severity. Proc Natl Acad Sci U S A 2021; 118 2021/08/27. DOI: 10.1073/pnas.2111315118.
12. Lim WW, Mak L, Leung GM, et al. Comparative immunogenicity of mRNA and inactivated vaccines against COVID-19. Lancet Microbe 2021; 2: e423. 2021/07/27. DOI: 10.1016/S2666-5247 (21) 00177-4.
13. Tun HM, Peng Y, Chen B, et al. Ethnicity Associations With Food Sensitization Are Mediated by Gut Microbiota Development in the First Year of Life. Gastroenterology 2021; 161:94-106. 2021/03/21. DOI: 10.1053/j.gastro.2021.03.016.
14. Lynn MA, Tumes DJ, Choo JM, et al. Early-Life Antibiotic-Driven Dysbiosis Leads to Dysregulated Vaccine Immune Responses in Mice. Cell Host Microbe 2018; 23:653-660 e655. 2018/05/11. DOI: 10.1016/j.chom.2018.04.009.
15. Oh JZ, Ravindran R, Chassaing B, et al. TLR5-mediated sensing of gut microbiota is necessary for antibody responses to seasonal influenza vaccination. Immunity 2014; 41:478-492. 2014/09/16. DOI: 10.1016/j.immuni.2014.08.009.
16. Pulendran B, P SA and O′Hagan DT. Emerging concepts in the science of vaccine adjuvants. Nat Rev Drug Discov 2021; 20:454-475. 2021/04/08. DOI: 10.1038/s41573-021-00163-y.
17. Kim M, Qie Y, Park J, et al. Gut Microbial Metabolites Fuel Host Antibody Responses. Cell Host Microbe 2016; 20:202-214. 2016/08/02. DOI: 10.1016/j.chom.2016.07.001.
18. Hedlund M, Frendeus B, Wachtler C, et al. Type 1 fimbriae deliver an LPS-and TLR4-dependent activation signal to CD14-negative cells. Mol Microbiol 2001; 39:542-552. 2001/02/13. DOI: 10.1046/j. 1365-2958.2001.02205.x.
19. Tartof SY, Slezak JM, Fischer H, et al. Effectiveness of mRNA BNT162b2 COVID-19 vaccine up to 6 months in a large integrated health system in the USA: a retrospective cohort study. Lancet 2021 2021/10/08. DOI: 10.1016/S0140-6736(21)02183-8.
20. Huda MN, Lewis Z, Kalanetra KM, et al. Stool microbiota and vaccine responses of infants. Pediatrics 2014; 134: e362-372. 2014/07/09. DOI: 10.1542/peds.2013-3937.
21. Strazar M, Mourits VP, Koeken V, et al. The influence of the gut microbiome on BCG-induced trained immunity. Genome Biol 2021; 22:275. 2021/09/24. DOI: 10.1186/s13059-021-02482-0.
22. Zhao T, Li J, Fu Y, et al. Influence of gut microbiota on mucosal IgA antibody response to the polio vaccine. NPJ Vaccines 2020; 5: 47. 2020/06/23. DOI: 10.1038/s41541-020-0194-5.
23. Huda MN, Ahmad SM, Alam MJ, et al. Bifidobacterium Abundance in Early Infancy and Vaccine Response at 2 Years of Age. Pediatrics 2019; 143 2019/01/25. DOI: 10.1542/peds.2018-1489.
24. Pifferi C, Fuentes R and Fernandez-Tejada A. Natural and synthetic carbohydrate-based vaccine adjuvants and their mechanisms of action. Nat Rev Chem 2021:1-20. 2021/02/02. DOI: 10.1038/s41570-020-00244-3.
25. Di Luccia B, Ahern PP, Griffin NW, et al. Combined Prebiotic and Microbial Intervention Improves Oral Cholera Vaccination Responses in a Mouse Model of Childhood Undernutrition. Cell Host Microbe 2020; 27:899-908 e895. 2020/04/30. DOI: 10.1016/j.chom.2020.04.008.
26. Yamamoto S, Mizoue T, Tanaka A, et al. Sex-associated differences between body mass index and SARS-COV-2 antibody titers following the BNT162b2 vaccine among 2,435 healthcare workers in Japan. medRxiv 2021:2021.2008.2030.21262862. DOI: 10.1101/2021.08.30.21262862.
27. Perera RA, Mok CK, Tsang OT, et al. Serological assays for severe acute respiratory syndrome coronavirus 2 (SARS-COV-2), March 2020. Euro Surveill 2020; 25 2020/04/30. DOI: 10.2807/1560-7917.ES.2020.25.16.2000421.
28. Perera R, Ko R, Tsang OTY, et al. Evaluation of a SARS-COV-2 Surrogate Virus Neutralization Test for Detection of Antibody in Human, Canine, Cat, and Hamster Sera. J Clin Microbiol 2021; 59 2020/11/4. DOI: 10.1128/JCM.02504-20.
29. Guittar J, Shade A and Litchman E. Trait-based community assembly and succession of the infant gut microbiome. Nat Commun 2019; 10:512. 2019/02/3. DOI: 10.1038/s41467-019-08377-w.
30. Zhang X and Yi N. NBZIMM: negative binomial and zero-inflated mixed models, with application to microbiome/metagenomics data analysis. BMC Bioinformatics 2020; 21:488. 2020/11/01. DOI: 10.1186/s12859-020-03803-z.

What is claimed is:

1. A method of enhancing antibody response in a human subject receiving an inactivated COVID-19 vaccine, comprising introducing into the subject's gastrointestinal tract a composition comprising an effective amount of (i) bacterial species Bifidobacterium adolescentis, or (ii) one or more of the bacterial species set forth in Table 1.

2. A method of enhancing antibody response in an obese or overweight human subject receiving an inactivated COVID-19 vaccine, comprising introducing into the subject's gastrointestinal tract a composition comprising an effective amount of one or more of the bacterial species set forth in Table 2.

3. The method of claim 2, wherein the composition further comprises an effective amount of one or more of the bacterial species set forth in Table 1.

4. A method of enhancing antibody response in a human subject receiving an mRNA COVID-19 vaccine, comprising introducing into the subject's gastrointestinal tract a composition comprising an effective amount of (i) bacterial species Bifidobacterium adolescentis; (ii) bacterial species Roseburia faecis; (iii) one or more of the bacterial species set forth in Table 3 or 4; or (iv) menaquinols.

5. A method of enhancing antibody response in an obese or overweight human subject receiving an mRNA COVID-19 vaccine, comprising introducing into the subject's gastrointestinal tract a composition comprising an effective amount of one or more of the bacterial species set forth in Table 5.

6. The method of claim 5, wherein the composition further comprises an effective amount of one or more of the bacterial species set forth in Table 3 or 4.

7. The method of claim 5 or 6, wherein the composition comprises an effective amount of one or more of the bacterial species set forth in Tables 3, 4, and 5.

8. The method of any one of claims 5-7, wherein the vaccine is BioNTech vaccine.

9. A method of reducing adverse effects in a human subject receiving an inactivated COVID-19 vaccine, comprising introducing into the subject's gastrointestinal tract a composition comprising an effective amount of (i) one or more of the bacterial species Prevotella copri, Megamonas funiformis, and Megamonas hypermegale; or (2) one or more of the bacterial species set forth in Table 6.

10. The method of any one of claims 1-3 and 9, wherein the vaccine is Sino Vac-Corona Vac.

11. A method of reducing adverse effects in a human subject receiving an mRNA COVID-19 vaccine, comprising introducing into the subject's gastrointestinal tract a composition comprising an effective amount of one or more of the bacterial species Prevotella copri, Megamonas funiformis, and Megamonas hypermegale.

12. The method of claims 11, wherein the vaccine is BioNTech vaccine.

13. The method of any claims 1-12, wherein the introducing step comprises delivery of the composition to the small intestine, ileum, or large intestine of the subject.

14. The method of claim 13, wherein a prebiotic or therapeutic agent for COVID-19 is introduced concurrently.

15. The method of claim 13, wherein the introducing step comprises oral ingestion of the composition.

16. The method of any one of claims 13-15, wherein the composition is in the form of a powder, liquid, paste, cream, tablet, or capsule.

17. The method of claim 13, wherein the introducing step comprises direct deposit of the composition to the subject's gastrointestinal tract.

18. The method of any one of claims 1-17, wherein the subject has received the vaccine within the past 24-48 hours or is to receive the vaccine within the next 24-48 hours.

19. A composition for use in enhancing immunity or reducing adverse effects from COVID-19 vaccination in a subject comprising an effective amount of (1) one or more bacterial species selected from Tables 1, 2, 5, and 6, Bifidobacterium adolescentis, and Prevotella copri; and (2) a physiologically acceptable excipient.

20. The composition of claim 19, further comprising one or more of bacterial species selected from Table 3 or 4.

21. The composition of claim 19 or 20, consisting essentially of an effective amount of (1) one or more of the bacterial species; (2) one or more physiologically acceptable excipients.

22. The composition of any one of claims 19-21, which is formulated for oral ingestion.

23. The composition of claim 22, which is in the form of a food or beverage item.

24. The composition of any one of claims 19-21, which is formulated for direct deposit to the subject's gastrointestinal tract.

25. The composition of any one of claims 19-24, further comprising a prebiotic or therapeutic agent for COVID-19.

26. A kit for enhancing efficacy or reducing adverse effects from COVID-19 vaccination comprising a plurality of compositions each comprising an effective amount of one or more bacterial species selected from Tables 1, 2, 5, and 6, Bifidobacterium adolescentis, Roseburia faecis, Prevotella copri, Megamonas funiformis, and Megamonas hypermegale.

27. The kit of claim 26, further comprising one or more compositions each comprising an effective amount of one or more different bacterial species selected from Table 3 or 4.

28. The kit of claim 26 or 27, further comprising one or more compositions each comprising an effective amount of a prebiotic or therapeutic agent for COVID-19.

29. The kit of any one of claims 26-28, wherein the compositions are in the form of a powder, liquid, paste, cream, tablet, or capsule.

30. The method of any one of claims 1-18, the composition of any one of claims 17-23, or the kit of any one of claims 26-29, wherein the composition comprises no detectable amount of another Bifidobacterium species.
